(19) United States
(12) Patent Application Publication
(10) Pub. No.: US 2005/0158370 A1
(43) Pub. Date: Jul. 21, 2005

(54) Charge-balanced chemoselective linkers

(75) Inventors: Nicholas Sean Flinn, Cambridge (GB); Martin Quibell, Cambridge (GB); William Gordon Turnell, Cambridge (GB); Manoj Kumar Ramjee, Cambridge (GB)

Correspondence Address: WILMER CUTLER PICKERING HALE AND DORR LLP, 60 STATE STREET, BOSTON, MA, 02109, US

(73) Assignee: Amura Therapeutics Limited, Cambridge (GB)

(21) Appl. No.: 10/510,893

(22) PCT Filed: Apr. 7, 2003
(86) PCT No.: PCT/GB03/01505

(30) Foreign Application Priority Data
Apr. 8, 2002 (GB) 0208061.2
Jul. 16, 2002 (GB) 0216516.5

(51) Int. Cl. A61L 15/00; C07D 209/18; C07C 237/22

(52) U.S. Cl. 424/445; 548/495; 562/450

(57) ABSTRACT

Compounds according to general formulae (Ia to Ie) wherein: X═O or S; Y is O, S or CH₂, CHR, CRR, where R is C₁₋₇ alkyl; Z is O or S; R₁ is H or C₁₋₇ alkyl; R₂ is H or C₁₋₇ alkyl; R₄ is H or C₁₋₇ alkyl at any vacant position on the aromatic ring; R₃ is C₁₋₇ alkyl-L₁-R₅-L₂-R₆—COOH, C₃₋₁₀ cycloalkyl-L₁-R₅-L₂-R₆—COOH or Ar—C₀₋₇ alkyl-L₁-R₅-L₂-R₆—COOH; each of L₁ and L₂ is absent or a suitable linker such as an amide CONH; or an ether —O—, or a thioether —S— or a sulphone —SO₂—; R₅ is C₁₋₇ alkyl, C₃₋₁₀ cycloalkyl or Ar—C₀₋₇ alkyl each of which is substituted with either NR₈R₉, where the nitrogen atom is capable of being protonated in solution to give N⁺HR₈R₉; or a quaternary nitrogen atom N⁺R₈R₉R₁₀, such that R₅ contains a positive charge; each of R₈, R₉ and R₁₀ is independently C₁₋₇ alkyl, C₃₋₁₀ cycloalkyl or Ar—C₀₋₇ alkyl, or any two or more of R₈, R₉ and R₁₀ together form an alicyclic or arylalicyclic ring system; R₆ is C₁₋₇ alkyl, C₃₋₁₀ cycloalkyl or Ar—C₀₋₇ alkyl; and their salts, hydrates, solvates, complexes or prodrugs are of use as linkers for conjugating an epitope to a carrier protein.

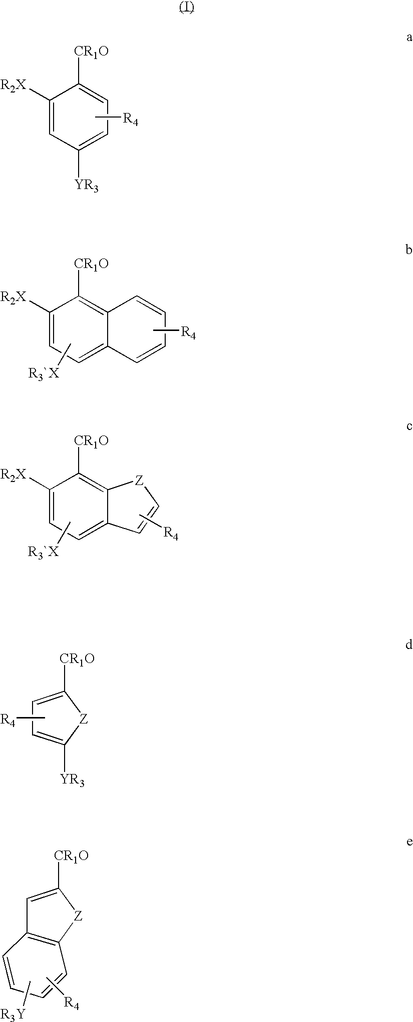

THE PRESENT INVENTION relates to constructs in which a plurality of active moieties are attached to a carrier, for example a protein, a glass slide or a polymeric surface and to linkers useful in the formation of such constructs. In particular, the invention relates to constructs having a chemoselective and selected quantifiable degree of loading. For example, high loading soluble protein constructs are provided, in which the active moieties, or epitopes, are linked to the carrier via a linker. The linkers also form a part of the invention and are selected such that the chemical point of reaction between the active moiety and carrier is chemoselectively controlled and the charge pattern at the surface of the loaded carrier closely resembles that of the unloaded carrier.

### BACKGROUND

The covalent chemical attachment of an active moiety to a carrier is a fundamental process that lies at the heart of a diverse range of scientific disciplines. For example, the attachment of peptides, oligosaccharides, DNA or small bioactive organic molecules to glass slides or chips has given rise to the enormous field of diagnostic screening, with example applications such as toxicological testing of new chemical entities and genetic profiling. Attachment of the same type of molecules to polymeric surfaces has applications in diverse fields such as affinity purification of small molecules/proteins and smart wound dressings that elicit a physiological response to enhance the wound healing process. Alternatively, attachment of such molecules to immunostimulatory proteins has application in the field of synthetic vaccine development.

Within each of these fields, successful application is dependent upon the stringent control of a number of key chemical and physiochemical parameters being achieved.

A key objective is to obtain quantitative and qualitative control of the covalent attachment chemistry since this should provide a final construct that exhibits an optimal combination of molecular display and physiochemical characteristics. Each application has many subtle variations of these key requirements to consider, given the range of chemical diversity and intrinsic characteristics present in active moieties such as peptides, oligosaccharides, DNA or small bioactive organic molecules and the different physiochemical properties of a glass slide when compared to a polymeric bead or a protein.

In the development of synthetic vaccines displaying peptidic epitopes, for example, a number of subtle variations need to be considered as will be described below.

Peptides identified as epitopes for vaccine development usually require conjugation to carrier proteins to provide a construct with which to provoke an immune response to the low molecular weight immunogen in vivo. This is illustrated in Scheme 1 below where an epitope (1) is reacted with a conjugate (2) and a carrier protein (3) to give a construct (4). An ideal vaccine construct would contain a high surface coverage of conjugated epitope on the carrier protein, whilst retaining high aqueous solubility. Additionally, the linkage created between the carrier protein and epitope ideally would be immunogenically inert and not involve residues or functionalities critical to epitope recognition (Briand. J. P., Muller, S. and Van Regenmortal, M. H. V. J. Immunol. Methods 78, 59-69, 1985). Presently, the most commonly used methods of conjugation in the preparation of experimental vaccines involve chemically non-specific reactions with glutaraldehyde (Avrameas, S. and Ternynck, T. Immunochemistry 6, 53, 1969; Korn, A. H., Feairheller, S. H. and Filachione, E. M. J. Mol. Biol. 65, 525, 1972; Reichlin, M. in: Methods in Enzymology, vol. 70, eds. Van Vunakis, H. and Langone, J. J. [Academic Press, New York] pp. 159-165, 1980), carbodiimides (Goodfriend, T. L., Levine, L. and Fasman, G. D. Science 144, 1344, 1964; Bauminger, S. and Wilchek, M. in: Methods in Enzymology, vol. 70, eds. Van Vunakis, H. and Langone, J. J. [Academic Press, New York] pp. 151-159, 1980), bis-diazotized benzidine (BDB) (Gordan, J., Rose, B. and Sehon, A. H. J Ex. Med. 108, 37, 1958) or utilise maleimide derivatives that rely on the presence of a thiol moiety (Liu, F.-T., Zinnecker, M., Hamaoka, T. and Katz, D. H. Biochemistry 18, 690, 1979; Yoshitake, S., Yamada, Y., Ishikawa, E. and Masseyeff, R. Eur. J. Biochem. 101, 395-399, 1979; Green, N., Alexander, H., Olson, A., Alexander, S., Shinnick, T. M., Sutcliffe, J. G. and Lerner, R. A. Cell 28, 477, 1982).

Since the chemical nature of the epitope, particularly in the field of peptide epitopes, is a rapidly advancing discipline, many of the above techniques are no longer fully compatible with the more advanced epitope chemistries. The introduction of improved structural characterisation and design elements such as the use of disulfide constrained peptide loops as structural mimics of the epitope in its native environment, require specific and acutely controlled methods of conjugation to ensure the chemical integrity of the loaded epitope. Here, existing conjugation methods suffer from a number of experimental difficulties (Scheme 1):

(a) Qualitative and quantitative assessment of loaded epitope from the construct, the sometimes chemically sensitive epitope having proceeded through a number of chemical processes, is problematic (Briand. J. P., Muller, S. and Van Regenmortal, M. H. V. J. Immunol. Methods 78, 59-69, 1985).

(b) Generally, as the surface loading of carrier proteins with the conjugated epitope increases, low and unpredictable solubility of the construct is observed (Qamar, S., Islam, M. and Tayyab, S. J. Biochem. 114, 786-792, 1993).

(c) Introduction of disulfide-bridged peptides is complicated by the presence of the thiol functionality required when using maleimide-based conjugation and may lead to disruption of the disulfide bond.

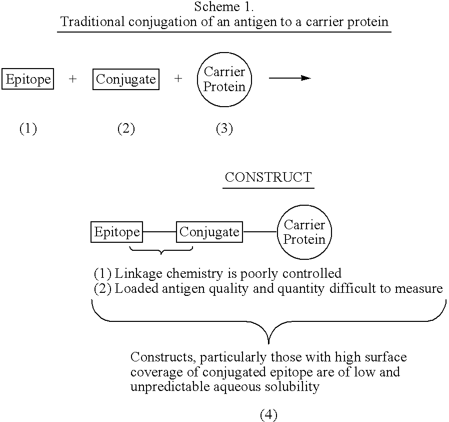

A number of the above issues have begun to be addressed in a new generation of constructs detailed in WO-A-0145745, which describes the invention of a process that allows the controlled linkage of a peptidic epitope to a carrier protein. The process provides a construct (7) from which the qualitative and quantitative assessment of epitope loading can be determined by simple chemical means as illustrated in Schemes 2&3.

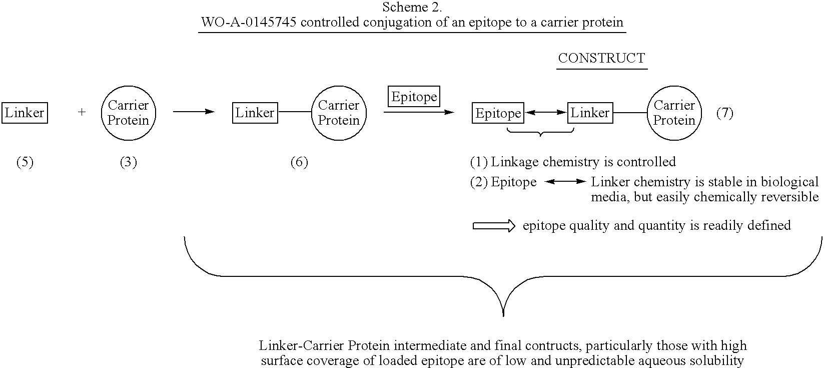

WO-A-0145745 describes a chemical linker (5) that contains a carboxylic acid and an aldehyde functionality. The carboxylic acid provides a point of attachment to the carrier protein by the formation of a secondary amide bond between the linker and the carrier protein accessible surface lysine residues—a process that yields an intermediate Linker-Carrier Protein (6). The aldehyde functionality provides a point of attachment to a peptidic epitope in a controlled and chemically reversible manner. Since the peptidic epitope itself may contain many chemically reactive functionalities (amino acid residue side chains containing amine, carboxylic acid, thiol, alcohol, imidazole, indole), controlled reaction to (6) is achieved through the chemoselective reaction of (6) with a hydrazide function, introduced into the epitope during synthesis (Scheme 3).

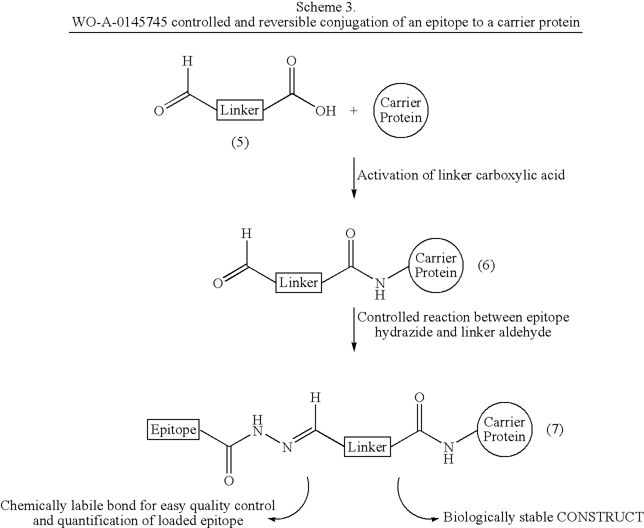

The hydrazide, being a weak base, forms a stable acyl-hydrazone bond with the aldehyde functionality in (6) at mildly acidic pH. At this pH, basic side chain nucleophiles on the epitope are protonated and excluded from the conjugation reaction (Jencks, W. P. J. Am. Chem. Soc. 81, 475-481, 1959; Reeves, R. L. in: The Chemistry of the Carbonyl Group, ed. Patai, S. (Interscience, London) pp. 600-614, 1966). Hydrazone formation has previously been employed in conjugation reactions via C-terminal hydrazides and N-terminal aldehydes that are traditionally generated by sodium metaperiodate mediated oxidation of an N-terminal serine residue within the specific proteins and peptides (King, T. P., Zhao, S. W. and Lam, T. Biochemistry 25, 5774-9, 1986; Rose, K., Vilaseca, L. A., Werlen, R., Meunier, A., Fisch, I., Jones, R. M. and Offord, R. E. Bioconj. Chem. 2, 154-159, 1991; Gaertner, H. F., Rose, K., Cotton, R., Timms, D., Camble, R. and Offord, R. E. Bioconj. Chem. 3, 262-268, 1992).

The process described in WO-A-0145745 offers a clear advance compared with previous methodologies due to the controlled nature of the conjugation procedure. This process allows a high level of construct quality control to be achieved, through chemical release and analytical characterisation of the intact epitope (1).

WO-A-0145745 has provided an impressive advance beyond previous methods. However, the whole question of construct solubility, an equally important consideration for the raising of antibodies and vaccination has not been addressed.

Widely used carrier proteins such as bovine serum albumin (BSA), ovalbumin and keyhole limpet haemocyanin (KLH) have a finely balanced surface distribution of charge. Conjugation of a linker/epitope preparation to these and other proteins disrupts the balance and distribution of charge within the protein and leads to an overall change in its isoelectric point (pI) and often results in conjugates with poor aqueous solubility at a relevant pH. As detailed in Scheme 3, each linker unit reacts with an accessible surface lysine residue to form a secondary amide bond, thus removing a positive charge from the carrier protein surface. As the surface coverage increases, a concomitant increase in unbalanced surface negative charge occurs, along with increasing steric hindrance within the construct (Ansari, A. A., Kidwai, S. A. and Salahuddin, A. J. Biol. Chem. 250, 1625-32, 1975). Conformational change is brought about as the levels of acylation increase, as shown in a previous study of the succinylation of BSA, in which a change of Stokes radius from 3.7 to 6.3 nm was observed upon 87% succinylation (Tayyab, S. and Qasim, M. A. Biochim. Biophys. Acta 913, 359-367, 1987). This may eventually lead to a destabilisation of the carrier protein tertiary structure and precipitation of the construct. Furthermore, because of the increase in net negative charge, the PI of the modified protein is reduced. Thus, as the pH of the solvent is lowered and approaches the new isoelectric point, the tendency of proteins to precipitate in the acidic media then becomes more likely (Shaw, K. L., Grimsley, G. R., Yakovlev, G. I., Makarov, A. A. and Pace, C. N. Prot. Sci. 10, 1206-15, 2001). This is particularly relevant to our preferred method of conjugation, since the final hydrazone bond formation between epitope and linker-carrier protein (6) is performed at acidic pH (down to pH 2.1) (Rose, K., Zeng, W., Regamey, P. O., Chernushevich, I. V., Standing, K. G. and Gaertner, H. F. Bioconj. Chem. 7, 552-556, 1996). Indeed in the case of WO-A-0145745 precipitation of the linker-carrier protein intermediate (6) was observed prior to attempted loading of epitope.

A major cause of construct insolubility at high surface coverage may be due to the build-up of unbalanced surface charge upon loading of epitope-linker. Modification of groups contributing negative charge may result in a net increase in the isoelectric point, whereas alteration of the positive charge bearing functions may result in net decrease in the isoelectric point of the protein. As the relationship between solubility and pH is a function of the isoelectric point of the protein, the ability to replace either positive or negative charge lost through chemical modification, provides an efficient way of controlling/improving the aqueous solubility of highly modified proteins. This necessitates the design and construction of a charge-balanced linker (8). Restoration of charge balance within the construct is conceptually simple and may be brought about, in our case, through the inclusion of an amine to replace that substituted during the conjugation (a negative charge may be replaced through the inclusion of functional groups containing a proton that readily dissociates e.g. hydroxyl or carboxylic acid moieties). However, this solution is not trivial, since the chemistry involved in the initial linker-carrier protein formation involves amide bond formation and as such, any amine functionality within the linker would need to be protected prior to and unmasked following the acylation of the carrier protein with the linker. A more pragmatic approach to charge-restoration would be through the introduction of a quaternary ammonium group. The quaternary nitrogen bears the positive charge, while remaining inert to further acylation.

The effect of quaternization on the solubility of proteins is well documented (Yamada, H., Seno, M., Kobayashi, A., Moriyama, T., Kosaka, M., Ito, Y. and Imoto, T. J. Biochem. 116, 852-857, 1994) and has also been employed to improve solubility of synthetic polymers and macromolecular constructs (Ishizu, K. and Kitano, H. J. Colloid Interface Sci. 229, 165-167, 2000; Thanou, M. M., Kotze, A. F., Scharringhausen, T., Luessen, H. L. de Boer, A. G., Verhoef, J. C. and Junginger, H. E. J Contr. Release 64, 15-25, 2000). Attachment of such a linker to the carrier protein will lead to a high loading and soluble, positive charge-balanced linker-carrier protein (9) (Scheme 4). In addition to the improved solubility characteristics, construct (9) could also be prepared as a core stock reagent, enabling uniform preparation of vaccine candidates and allowing a more precise comparison of different immunogens.

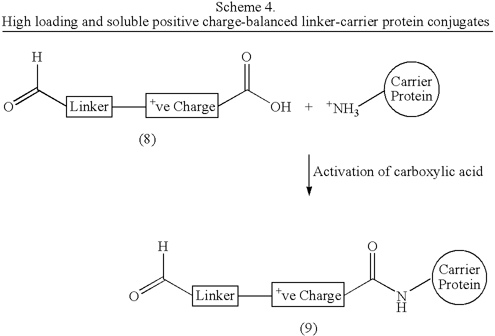

A second highly desirable attribute in the preparation of constructs is the real-time analytical monitoring and control of the conjugation process. In WO-A-0145745 monitoring was only achieved by the use of a chemically destructive release of the intact epitope from the construct. Clearly, it would be advantageous to monitor the rate and extent of construct formation without the necessity of breaking down the construct since such monitoring provides the possibility of controlling the conjugation process.

While the increased solubility of the constructs is mainly important in solution phase applications, the ability to monitor the rate and extent of a reaction is important both for solid and solution phase uses.

The design and preparation of a robust positive charge balanced linker (8) is a surprisingly demanding task, within which many interlinked properties need to be considered:—

(a) Ideally, the positive charge in (8) should be in close proximity to the carrier surface charge (for example the protein surface lysine charge) that is removed upon coupling, and provide a centre of comparable pKa.

(b) The preparation of (8) should be smooth and reproducible.

(c) Carboxylic acid activation of (8) prior to addition to the carrier (for example carrier protein) should proceed smoothly with minimal interference from the positive charge.

(d) Formation of the secondary amide bond between (8) and the carrier (for example carrier protein) should proceed smoothly with minimal interference from the positive charge.

(e) Formation of the acyl hydrazone bond between the active moiety-hydrazide (for example epitope-hydrazide) and positive charge balanced linker-carrier protein (9) should proceed smoothly with minimal interference from the positive charge.

(f) The reversible acid lability of the acyl hydrazone bond between the active moiety-hydrazide (for example epitope-hydrazide) and (9) requires the linker element of (8) to be an electron-rich aromatic moiety.

(g) The reversible acid lability of the acyl hydrazone bond between the active moiety-hydrazide (for example epitope-hydrazide) and (9) should not be adversely altered by the presence of the positive charge.

If the construct forming process is to be monitored using a destructive analytical technique, a critical theoretical design element relevant to the properties described in (a)→(g) is the retention of the reversible acid lability of the active moiety (epitope) acyl hydrazone bond in construct (10) since this allows analytical analysis of the loaded epitope hydrazide on construct (9) (Scheme 5).

However, if the aldehyde functionality within (9) is bonded to an appropriately derivatised moiety (R group), real-time analytical monitoring and control of the whole construct formation process may be addressed by analysis of the absorbance and fluorescence spectral differences between the carrier protein alone and species (9) and (10), at appropriate pH conditions.

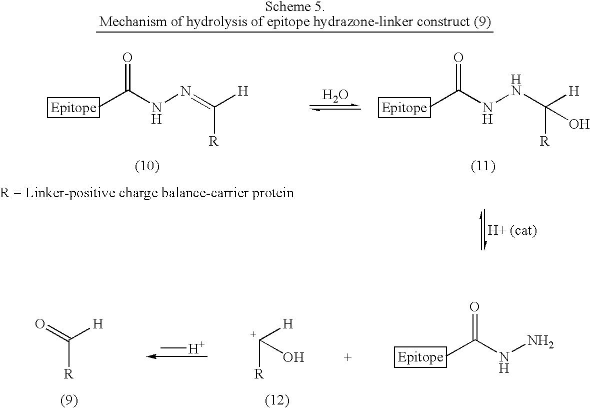

A mechanism for the acid catalysed hydrolysis of (10) proceeds through the addition of water to the carbon-nitrogen double bond giving (11). This would be followed by elimination of the epitope acyl hydrazide (hence readily available for analysis) and formation of an intermediate carbocation (12), then loss of a proton to give construct (9). The ease of this hydrolytic process will in part depend upon the resonance stabilisation of carbocation (12).

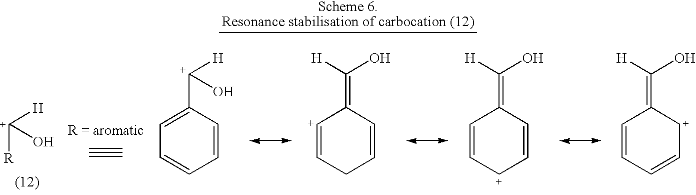

Resonance stabilisation of carbocation (12) is most readily achieved through an aromatic ring, preferably an electron-rich aromatic ring and more preferably a ring that contains π-electron-donating substituents situated ortho and para (see Scheme 6). Many examples exist in the literature describing the relationship between acid lability and substitution stereoelectronics for aromatic systems (e.g. see Johnson, T., Quibell, M. and Sheppard, R. C. J. Pept. Sci. 1, 11-25, 1995). Ortho and para alkoxy-type substituents are required on linker (8) so that the epitope-linker hydrazone bond is labile to 1N HCl or trifluoroacetic acid (TFA) i.e. relatively mild conditions that do not adversely affect peptidic epitopes and will allow easy hydrolysis and representative analysis of epitope from construct (9).

The structure shown above, in which the aldehyde functionality (9) is bonded to an electron rich aromatic ring has the additional advantage that it makes it possible to monitor the formation of the construct by comparing the absorbance and/or fluorescence spectra of the unassociated carrier with those of the species (9) and (10) at appropriate pH conditions. It is particularly useful if the aromatic ring is substituted with a group which ionises as a result of a change in pH. The ability to monitor the progress of the construct-forming reaction also makes it possible to control various aspects of the process, for example the degree of loading of the carrier with one or more active moieties.

Thus, accordingly, the first aspect of the invention provides a positive charge-balanced linker according to general formulae (Ia to Ie): 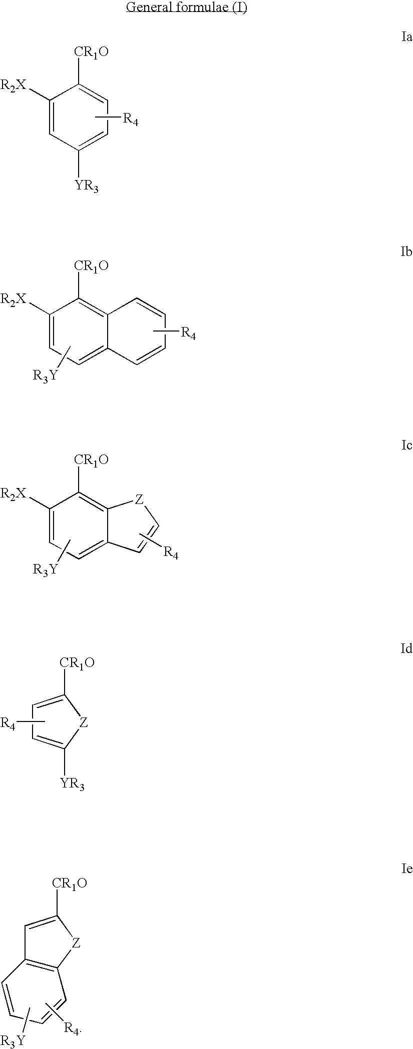
wherein:
X═O or S;
Y is O, S or CH₂, CHR, CRR, where R is C₁₋₇ alkyl;
Z is O or S;
R₁ is H or C₁₋₇ alkyl;
R₂ is H or C₁₋₇ alkyl;
R₄ is H or C₁₋₇ alkyl at any vacant position on the aromatic ring;
R₃ is C₁₋₇ alkyl-L₁-R₅-L₂-R₆—COOH, C₃₋₁₀ cycloalkyl-L₁-R₅-L₂-R₆—COOH or Ar—C₀₋₇ alkyl-L₁-R₅-L₂-R₆—COOH;
   each of L₁ and L₂ is absent or a suitable linker such as an amide CONH; or an ether —O—, or a thioether —S— or a sulphone —SO₂—;
   R₅ is C₁₋₇ alkyl, C₃₋₁₀ cycloalkyl or Ar—C₀₋₇ alkyl each of which is substituted with either NR₈R₉, where the nitrogen atom is capable of being protonated in solution to give N⁺HR₈R₉; or a quaternary nitrogen atom N⁺R₈R₉R₁₀, such that R₅ contains a positive charge;
      each of R₈, R₉ and R₁₀ is independently C₁₋₇ alkyl, C₃₋₁₀ cycloalkyl or Ar—C₀₋₇ alkyl, or any two or more of R₈, R₉ and R₁₀ together form an alicyclic or arylalicyclic ring system;
   R₆ is C₁₋₇ alkyl, C₃₋₁₀ cycloalkyl or Ar—C₀₋₇ alkyl;
or a salt, hydrate, solvate, complex or prodrug thereof.

Compounds of formula (I) can be reacted with a carrier to give a derivatised carrier in which the surface charge pattern is substantially the same as that of the original carrier. The carrier may be a protein. Because the charge pattern is substantially unchanged, it is possible to achieve high degrees of loading of active moieties onto a protein carrier without substantially compromising the solubility of the protein.

In addition, if the carrier is a polymeric surface or a glass slide, the linker derivatised carrier containing the charge-balance may exhibit beneficial solvation properties and/or a chaotropic effect that will enhance presentation of the loaded active moiety.

In addition to their increased solubility, the constructs formed using the charge balanced linkers of the present invention have the advantage that they can be used to load an active moiety onto a carrier in a controlled and chemoselective manner. The degree of loading can be selected to be optimal for the intended use of the construct and can be determined quantitatively for analysis purposes.

A further advantage is that the formation of constructs from the charge balanced linkers of the present invention may be monitored through the absorbance and fluorescence spectral differences between the unloaded carrier, linker derivatised carrier and the full hydrazone derivatised construct.

In the present specification, the term ‘heteroatom’ defines oxygen (O), sulphur (S) and nitrogen (N);

‘Halogen’ defines fluorine (F), chlorine (Cl), and bromine (Br).

‘C₁₋₇-alkyl’ as applied herein is meant to include stable straight or branched aliphatic saturated or unsaturated carbon chains containing one to seven carbon atoms such as methyl, ethyl, n-propyl, isopropyl, n-butyl, isobutyl, t-butyl, pentyl, isopentyl, hexyl, heptyl and any simple isomers thereof. Additionally, any C₁₋₇-alkyl may optionally be substituted at any point by one, two or three halogen atoms (as defined above) for example to give a trifluoromethyl substituent. Furthermore, C₁₋₇-alkyl may contain one or more heteroatoms (as defined above) for example to give ethers, thioethers, sulphones, sulphonamides, substituted amines, amidines, guanidines, carboxylic acids, carboxamides. If a heteroatom is located at a chain terminus then it is appropriately substituted with one or two hydrogen atoms. A heteroatom or halogen is only present when C₁₋₇-alkyl contains a minimum of two carbon atoms.

‘C₃₋₁₀-cycloalkyl’ as applied herein is meant to include any variation of ‘C₁₋₇-alkyl’, which additionally contains a 3 to 6 membered carbocyclic ring such as cyclopropyl, cyclobutyl, cyclopentyl, cyclohexyl. The carbocyclic ring may optionally be substituted with one or more halogens (as defined above) or heteroatoms (as defined above) for example to give a tetrahydrofuran, pyrrolidine, piperidine, piperazine or morpholine substituent.

‘Ar—C₀₋₇-alkyl’ as applied herein is meant to include any variation of C₁₋₇-alkyl which additionally contains an aromatic ring moiety ‘Ar’. The aromatic ring moiety Ar can be a stable 5 or 6-membered monocyclic or a stable 9 or 10 membered bicyclic ring which is unsaturated. The aromatic ring moiety Ar may be additionally substituted by any variation of C₁₋₇-alkyl. When C=0 in the substituent Ar—C₀₋₇-alkyl, the substituent is simply the aromatic ring moiety Ar.

The present invention includes all salts, hydrates, solvates, complexes and prodrugs of the compounds of this invention. Additionally, the present invention includes all isomers of stereochemical centres and all double bond isomers such as entgegen (E) or zusammen (Z) alkenes and syn or anti hydrazones. The invention also encompasses compounds incorporating other isotopes than the most common ones, for example isotopes of carbon, hydrogen, oxygen and nitrogen such as ¹⁴C, ²H, ¹⁷O and ¹⁵N.

In the context of the present invention, the term “active moiety” or “active moieties” refers to an epitope, a mimotope or a ligand. In the present invention, the active moieties will, if necessary, be derivatised in order to allow them to react in a chemically selective manner with the linker of general formula (I). Suitable derivatives for a chemically selective reaction include hydrazide analogues. Where the active moiety is a peptide, derivatisation towards a hydrazide may be achieved by reaction of a lysine side chain or N-terminal nitrogen or C-terminal carboxylic acid with a reagent to provide a hydrazide. If the active moiety does not include a suitable substituent for reaction to prepare a suitable derivative such as a hydrazide, it may be modified to introduce such a group, through for example an amine group. For example, sugars and oligosaccharides may be converted at the reducing end saccharide to the glycosylamine, via the Kochetkov reaction (Vetter, D. and Gallop, M. A. Bioconj. Chem. 6, 316-318, 1995). The glycosylamine may be converted into a hydrazide by trans-hydrazinolysis (see Prasad, A. V. N. and Richards, J. C. WO9702277). As a further example, oligonucleotides can be prepared that contain a modified base that contains a specific hydrazide functionality (see Strobel, H. et al, Nucl. Acids Res. 30(9) 1869-1878, 2002) or oligonucleotides can be prepared that contain a 2, 3 or 5-prime hydrazide moiety.

In some cases, more than one type of active moiety may be attached to a carrier.

The term “epitope” refers to a molecule which is capable of binding specifically to a biological molecule such as an antibody, antigen or cell surface receptor. The epitope may be a fragment, for example an antigenic determinant, derived from a carbohydrate, protein or peptide molecule or a variant or analogue of such a molecule. Examples of epitopes which can be used with this method include oxytocin and analogues thereof, B-cell and T-cell epitopes and antigenic determinants derived from a surface oligosaccharide from a pathogenic organism such as a bacteria

A “mimotope” is a synthetic molecule that mimics the activity of an epitope.

A “ligand” is a moiety that can bind to a receptor and elicit a response. A ligand may be a peptide, protein, sugar, lipid, nucleic acid, alkaloid, vitamin or a small organic molecule. For example, it may be an enzyme for use in an ELISA or in some other assay or a peptide growth factor or chemo-attractant protein suitable for use in a wound dressing. Other examples include proteins such as heparin. Alternatively, the ligand may be a labelling molecule such as a chromophore (biochemical, biophysical or chemical), fluorophore (biochemical, biophysical or chemical), luminophore (biochemical, biophysical or chemical), phosphorescence, radiochemical, quantum dot, electron spin tag, magnetic particle, nuclear magnetic resonance tag, x-ray tag, microwave tag, electrochemical, electrophysical (e.g. increased resistance), surface plasmon resonance, calorimetry, etc.

The “carrier” may be a proteinaceous molecule containing a plurality of active sites which react with a suitably derivatised epitope through a conjugation reaction. Examples of suitable carrier proteins include bovine serum albumin (BSA), ovalbumin and keyhole limpet haemocyanin (KLH), heat shock proteins (HSP), thyroglobulin, immunoglobulin molecules, tetanus toxoid, purified protein derivative (PPD), aprotinin, hen egg-white lysozyme (HEWL), carbonic anhydrase, ovalbumin, apo-transferrin, holo-transferrin, phosphorylase B, β-galactosidase, myosin, bacterial proteins and other proteins well known to those skilled in the art. Alternatively, the carrier may be chosen from large, slowly metabolised macromolecules such as polysaccharides, (sepharose, agarose, cellulose) cellulose beads, polymeric amino acids, polymers, including copolymers and some vitamins and alkaloids. Inactive virus particles (e.g. the core antigen of Hepatitis B Virus, see Murray, K. and Shiau, A-L., Biol. Chem, 380, 277-283, 1999) and attenuated bacteria such as Salmonella may also be used as carriers for the presentation of active moieties.

In solid supported applications, the term “carrier” may apply to an insoluble polymer such as a resin bead or plastic sheet or glass slide etc.

For certain aspects of the inventions, the terms “ligand” and “carrier” may be interchangeable in order to accommodate linking “ligand” to “ligand” and/or “carrier” to “carrier”.

“Conjugate” refers to a molecule which is capable of linking an epitope to a carrier protein in a chemically non specific manner.

“Linker” refers to a molecule which is capable of undergoing a specific chemical reaction with both a carrier and an active moiety such as an epitope so as to link the two together in a chemoselective (a selective reaction at a single functional group within a compound that contains multiple functional groups) manner.

“Construct” refers to a carrier linked to a plurality of active moieties via linkers or conjugates.

A “charge balanced linker” is a linker which is charged such that when it reacts with a carrier, the overall surface charge pattern of the carrier remains essentially unchanged.

A “positive charge balanced linker” is a charge balanced linker carrying a positive charge.

In the compounds of general formula (I), it is preferred that, independently or together:
X is oxygen;
Y is oxygen;
R₁ is hydrogen, methyl or ethyl, with hydrogen being particularly suitable;
R₂ is hydrogen or C₁₋₄ alkyl with more preferred compounds having R₂ as hydrogen, methyl or ethyl, particularly hydrogen or methyl and the most preferred being hydrogen;
L₁ is an amide CONH; and
L₂ is an amide CONH.

Within the definition of R₃, the positive nitrogen atom in R₅ needs to be an appropriate distance from the aromatic ring such that it does not adversely interfere with the aromatic ring electronics and hence ability to resonance stabilise carbocation (12). Additionally, in order to facilitate synthesis from readily available starting reagents and incorporation of R₅, preferred R₃ substituents in general formula (II) are chosen from simple (i.e. unsubstituted) straight chain alkyl groups or simple cycloalkyl groups or simple aromatics containing a carboxylic acid. Particularly suitable cycloalkyl groups in R₃ are those which include a cyclopentyl or cyclohexyl moiety, while examples of aromatic groups include phenyl, alkyl phenyl (for example benzyl) or phenyl alkyl. Specific examples of suitable R₃ groups are: 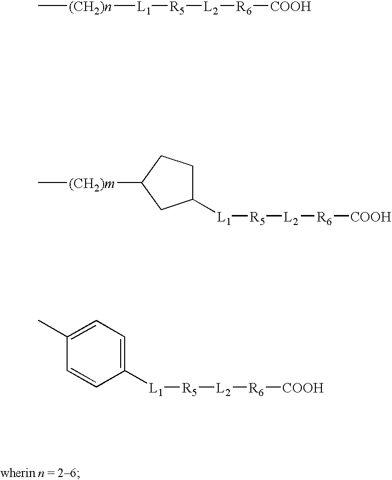
wherein
n=2-6;
m=1-3.

A preferred definition of R₅ provides a positive nitrogen atom that resembles as closely as possible the properties of the protein surface lysine residue that it is designed to mimic. Additionally, in order to facilitate the incorporation of R₅ within the framework detailed in general formula (I) from readily available starting reagents, it is preferred that the substituents NHR₅CO (where the NH is part of the L₁ moiety and the CO is part of the L₂ moiety) are chosen from simple amino acid residues that contain a side-chain protonatable amine functionality.

Also preferred in the definition of NHR₅CO is an amino acid residue that, through linker (8), directly incorporates the charge-balance to the carrier protein. Thus, a high loading and soluble positive charge-balanced linker-carrier protein (9) results, which otherwise through the addition of a protected amine functionality in R₅ would provide an intermediate construct (9) containing a latent amine functionality and suffer from the previously described low solubility problems.

Suitable amino acid residues for NHR₅CO may be represented by the formula:
—NH—CH[(CH₂)ₚN⁺R₈R₉R₁₀]CO—
wherein p is 1 to 5 (preferably 1 to 4 and more preferably 1 to 3) and R₈, R₉ and R₁₀ are as defined above.

For ease of synthesis and in order to avoid steric hindrance, the more suitable R₈, R₉ and R₁₀ groups include C₁₋₄ alkyl, with methyl being particularly preferred.

Within the definition of R₃, the substituent R₆ is defined as a spacer and is required to enable the smooth activation of linker (8) prior to formation of the positive charge-balanced linker-carrier protein (9). It is well known in the art of peptide chemistry that the activation of a non-urethane protected amino acid can lead to racemisation of the Cα-chiral centre (e.g. see Benoiton, N. L. and Kuroda, K. Int. J. Pept. Prot. Res. 17, 197, 1981). Also, it is well known in the art of peptide chemistry that the activation of the non side-chain protected amino acids which are preferred for R₅ requires special conditions and often result in unwanted side reactions. Taking these considerations into account, the spacer R₆ is required to alleviate the above potential difficulties and provide an easily activated carboxylic acid functionality.

It is preferred that R₆ combines with an NH group derived from the L₂ moiety and the terminal COOH to form an amino acid residue of the formula:
—NH—(CH₂)_{q}-Aₛ-(CH₂)ᵣCOOH;
where q and r are each 0 to 3, provided that both q and r are not both 0;
s is 0 or 1; and
A is a 5-10 membered stable monocyclic or bicyclic aromatic ring or a 3-6 membered carbocyclic or alicyclic ring.

It is more preferred that r and s are 0 and q is 1 or 2.

To facilitate synthesis of linker (8), routes commencing from readily available starting reagents are preferred. Thus compounds of general formulae (Ia) are preferred, particularly linkers designed around a 2,4-dialkoxy substituted benzaldehyde as defined in general formula (II): 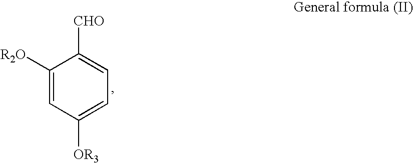
which is a compound of general formula (Ia) in which X and Y are O, R₁ is H and R₂ and R₃ are as defined above.

A more preferred embodiment of the compound of general formula (II) is detailed by general formula (III): 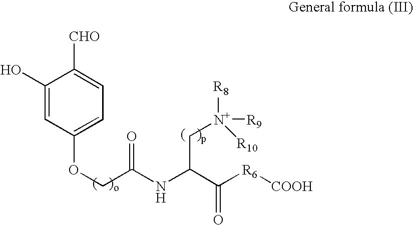
wherein:
o is an integer from 2-6;
p is an integer from 1 to 5 (preferably from 1 to 4 or even 1 to 3); and

R₆, R₈, R₉ and R₁₀ are as defined above.

In the embodiment of general formula (III), the combination NH—R₅CO (where NH forms part of the L₁ moiety and CO forms part of the L₂ moiety) is represented by an amino acid residue which contains a side chain with a quaternary nitrogen atom. The NH—R₅CO group can therefore replace the charge of a side chain lysine on a carrier protein which reacts with the carboxylic acid group attached to R₆.

A still more preferred embodiment of the compound of general formula (III) is detailed in general formula (IV): 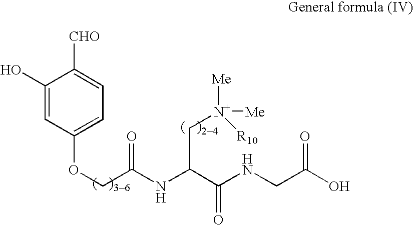
wherein R₁₀=Me or R₁₀=“−”, where the nitrogen may quaternise by protonation.

Compounds of general formula (I) in which L₁ and L₂ are CONH can be synthesised either in solution or on the solid phase. Compounds of general formula (I) in which L₁ and L₂ are CONH can be synthesised on the solid phase by:
(i) reacting a compound of general formula V:
   H₂N—R₆—COOH (V)
   wherein R₆ is as defined for general formula (I); and
wherein the compound of general formula (V) is bound at its C-terminus to a solid support;
with a compound of general formula (VI):
   W—NH—R₅—COOH (VI)
   wherein:
   R₅ is as defined for general formula (I); and
   W is a protecting group.
(ii) removal of the protecting group W and reaction with a compound of general formula (VII): 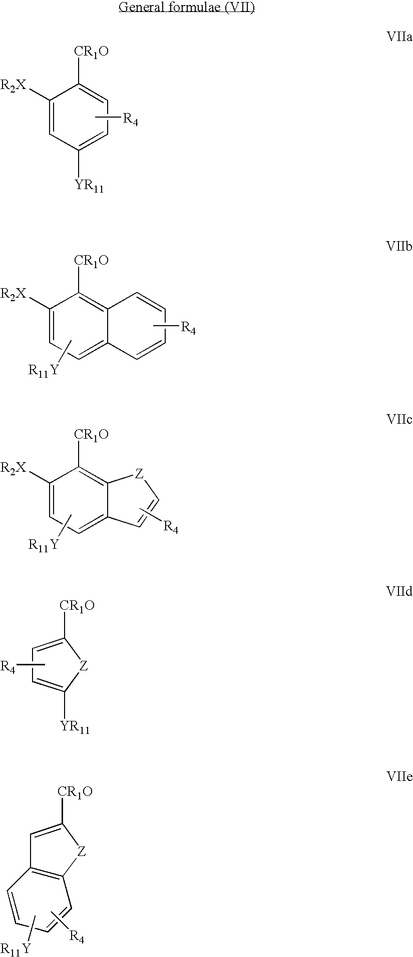
   wherein
   X, Y, Z, R₁, R₂ and R₄ are as defined for general formula (I); and
   R₁₁ is C₁₋₇ alkyl-COOH, C₃₋₁₀ cycloalkyl-COOH or Ar—C₀₋₇ alkyl-COOH; and
(iii) removal of the product from the solid support.

Suitable solid supports for use in the method include any resins suitable for the synthesis of peptide carboxylic acids such as 2-chlorotrityl resin. Removal from chlorotrityl resin can be achieved by treating the product with an acid, for example trifluoroacetic acid in a polar organic solvent such as dichloromethane.

The protecting group W is a urethane protecting group e.g. a group such as Fmoc (see Atherton, E and Sheppard, R. C. in ‘Solid Phase Peptide Synthesis: A Practical Approach’, IRL Press, 1989. for a thorough description of solid phase synthesis via the 9-fluorenylmethoxycarbonyl (Fmoc) protection strategy) which can be removed when required by treatment with piperidine in dimethylformamide.

Alternatively, compounds of general formula (I) can be prepared from compounds of general formulae (V), (VI) and (VII) by traditional solution phase peptide chemistry methods well known to those skilled in the art.

Compounds of general formulae (V), (VI) and (VII) are readily available and are well known to those of skill in the art.

In general, it is preferred that L₁ is an amide CONH and L₂ is an amide CONH, primarily due to the ready availability of amino acid reagents. However, non amide L₁ and L₂ containing linkers may also provide the chemoselective, quality control and charge balance properties through, for example, compounds of general formulae (VIII):

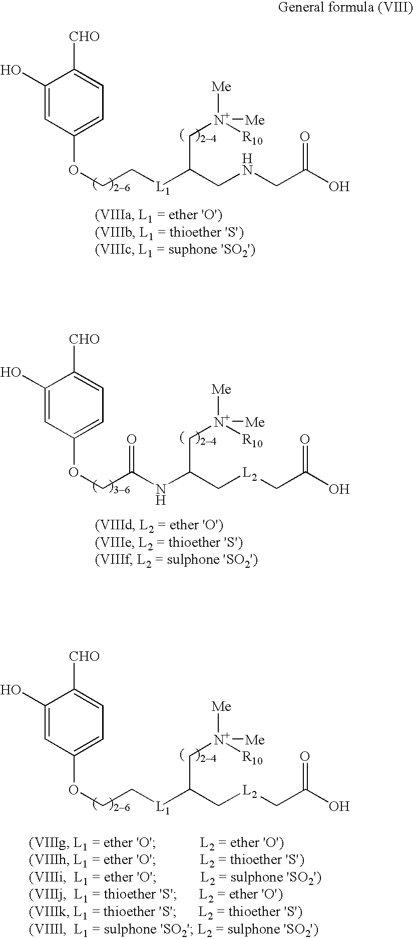

General synthesis of ethers, thioethers and sulphones in solution and on the solid phase are well known to those skilled in the art (e.g. see (a) Degerbeck, F. et al, J. Chem. Soc, Perkin Trans. 1, 11-14, 1993. for conversion of amino acids into α-hydroxyacids; (b) Souers, A. J. et al, Synthesis, 4, 583-585, 1999. for conversion of aminoacids into α-bromoacids; (c) Grabowska, U. et al, J Comb. Chem., 2(5), 475-490, 2000, for solid phase syntheses). For example, treatment of an α-hydroxyacid with sodium hydride and an alkyl halide provides an ether. Alternatively, treatment of an α-bromoacid with an alkyl thiol provides a thioether. Thioethers may be readily oxidised to provide sulphones. Combinations of these basic chemical reactions may be used to provide compounds of general formulae (VIII). An example synthesis towards a compound of formula (VIIIb) is detailed in Scheme 6;

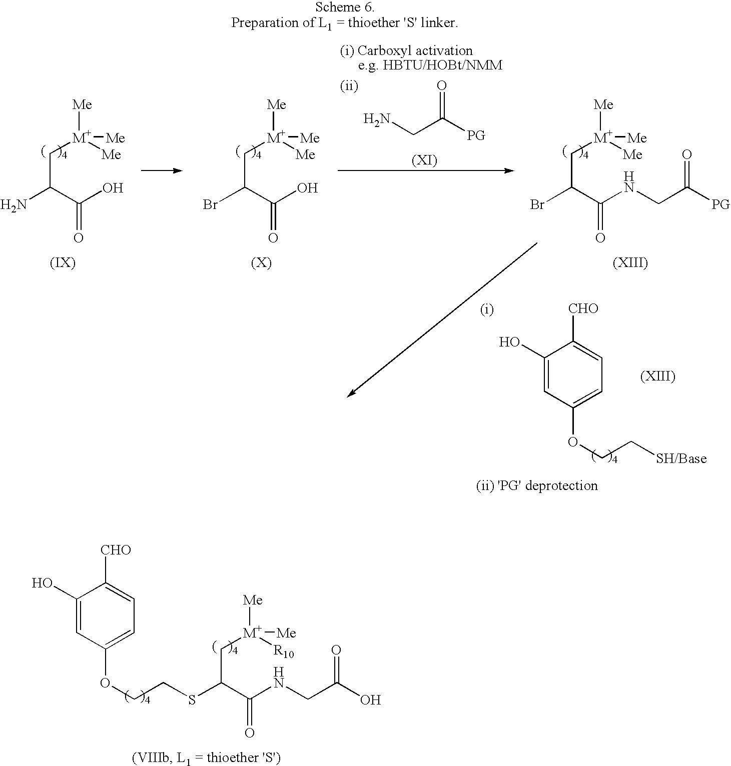

Treatment of amino acid (In) with sodium nitrite/H₂SO₄/potassium bromide provides the α-bromoacid (X) (Souers, A. J. et al, Synthesis, 4, 583-585, 1999) with retention of configuration. Coupling of carboxyl activated α-bromoacid (X) to the free amino of a carboxyl protected glycine (XI) provides building block (XII). Typical carboxyl protecting groups well known to those skilled in the art may be used such as tert-butyl ester or for solid phase syntheses groups such as the 2-chlorotrityl ester. Nucleophilic displacement of bromide (XII) with thiol (XIII) with base catalysis proceeds with inversion of configuration. Removal of the carboxyl protection (e.g. 95% aq trifluoroacetic acid where ‘PG’=tert-butyl ester) provides linker (VIIIb) which may be utilised in a similar manner to compounds of general formula (IV). As discussed in detail above, compounds of general formula (I) are of use for linking active moieties such as epitopes to carriers, for example proteins.

Therefore, in a further aspect of the invention, there is provided a compound of general formula (XIV): 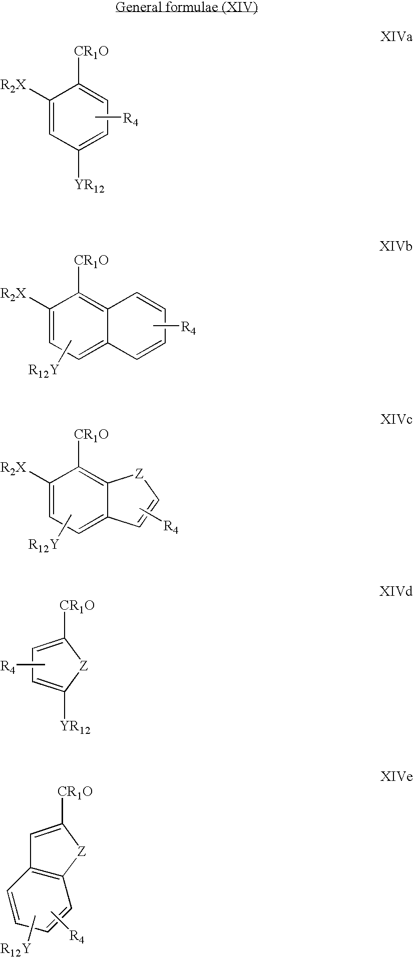
wherein
X, Y, Z, R₁, R₂ and R₄ are as defined for general formula (I); and
R₁₂ is C₁₋₇ alkyl-L₁-R₅-L₂-R₆CONHQ, C₃₋₁₀ cycloalkyl-L₁-R₅-L₂-R₆CONHQ or Ar—C₀₋₇ alkyl-L₁-R₅-L₂-R₆CONH-Q;
   wherein L₁, L₂, R₅ and R₆ are as defined in general formula (I);
Q is a residue which is part of a carrier and which either contains groups from which the “NH” moiety in R₁₂ is derived or has been derivatised so as to include such groups;
   wherein the carrier may contain multiple Q residues that already have 0,1,2, . . . nn linker molecules of general formula (I) attached;
   wherein the integer nn is the total number of Q residues available for attachment of a linker molecule to a specific carrier, where nn will be different for each specific carrier.

The carrier may be a proteinaceous molecule and in this case Q and the NH moiety in R₁₂ may be derived from a lysine side chain.

Suitable carrier proteins include bovine serum albumin, keyhole limpet haemocyanin (KLH), ovalbumin, heat shock proteins (HSP), thyroglobulin, immunoglobulin molecules, tetanus toxoid, purified protein derivative (PPD), aprotinin, hen egg-white lysozyme (HEWL), carbonic anhydrase, ovalbumin, apo-transferrin, holo-transferrin, phosphorylase B, β-galactosidase, myosin, bacterial proteins, inactive virus particles (e.g. the core antigen of Hepatitis B Virus, see Murray, K. and Shiau, A-L., Biol. Chem, 380, 277-283, 1999) and other proteins well known to those skilled in the art.

Non-protein carriers include large, slowly metabolised macromolecules such as polysaccharides (sepharose, agarose, cellulose), cellulose beads, polymeric amino acids, copolymers, inactive virus particles and attenuated bacteria such as Salmonella may also be used as carriers for the presentation of active moieties.

The invention further comprises a process for the preparation of a compound of general formula (XIV) as defined above, the process comprising reacting a compound of general formula (I) as defined above with a carrier, such as a protein.

The reaction can be achieved by reacting a solution or suspension of the carrier in an aqueous solvent with a compound of formula (I) or a derivative thereof, for example the succinimide ester, symmetrical or unsymmetrical anhydride, maleimide, or an acid fluoride or chloride, a pentafluorophenol ester, or other active ester known to those skilled in the art, in a solvent such as dimethyl sulfoxide at a temperature of from 15 to 50° C., but preferably at room temperature. The reaction may be conducted at a pH greater than 7.

Compounds of general formula (XIV) are intended for linkage to a derivatised active moiety such as an epitope or a ligand and therefore, in a further aspect of the invention, there is provided a compound of general formula (XV): 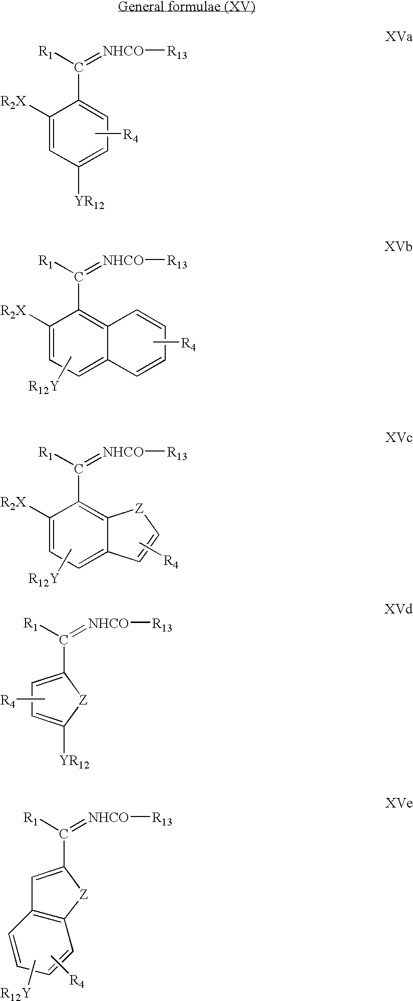
wherein X, Y, Z, R₁, R₂ and R₄ are as defined for general formula (I);
R₁₂ is as defined in general formula (XIV);
R₁₃ is (CH₂)ₜCONH-E, CONH-E or G;
   t is an integer from 1 to 5;
   E is derived from an active moiety which either contains an amino group or has been derivatised to do so; and NHE is derived from the amino group of the active moiety;
   G is an active moiety bound to the carbonylhydrazide through a carbon atom

When the active moiety from which E is derived is a peptide, the amino group may be derived from a side-chain lysine or N-terminal amine.

The compound of formula XV may comprise groups E and/or G derived from two or more active moieties. This can be particularly useful in applications such as raising antibodies to an epitope or mimotope, e.g. where both a T-cell and B-cell epitope may be attached to each carrier protein, or an adjuvant can also be linked to a carrier, or in analytical methods where a probe and a marker can both be linked to the carrier.

The compounds of general formula (XV) are simple to prepare from compounds of general formula (XIV) and thus, in a further aspect of the invention, there is provided a process for the preparation of a compound of general formula (XV) as defined above, the process comprising reacting a compound of general formula (XIV) as defined above with a compound of general formula (XVIa), (XVIb) or (XVIc):
E-NH—CO—(CH₂)ₜCONHNH₂ (XVIa)
E-NH—CO—NHNH₂ (XVIb)
G-CO—NHNH₂ (XVIc)
where E, G and t are as defined above.

The reaction may be carried out in an aqueous or a hydrophilic organic solvent at a temperature of from 15 to 50° C. but preferably at room temperature.

Compounds of general formula (XVIa) can be prepared from an active moiety such as an epitope by chemoselective reaction (a selective reaction at a single functional group within a compound that contains multiple functional groups) between a side chain of an epitope lysine residue or an N-terminal amine group with a compound of the formula (XVIIa):
HOOC—(CH₂)ₜCONHNH-J (XVIIa)

Where t is as defined above and J is a protecting group such as Boc (tert-butoxycarbonyl) or Fmoc (9-fluorenylmethoxycarbonyl).

The carboxylic acid of compound (XVIIa) is activated such as the succinimide ester, symmetrical or unsymmetrical anhydride, maleimide, or an acid fluoride or chloride, a pentafluorophenol ester, or other active ester known to those skilled in the art and reacted with a side chain of an epitope lysine residue or an N-terminal amine group. Chemoselective reaction of a side chain of an epitope lysine residue or an N-terminal amine group with a compound of formula (XVIIa) is achieved by reaction of an otherwise fully protected epitope (see Atherton, E and Sheppard, R. C. in ‘Solid Phase Peptide Synthesis: A Practical Approach’, IRL Press, 1989. for a thorough description of side-chain protection strategy) containing a single free side-chain lysine residue or N-terminal amine group. An otherwise fully protected epitope containing a single free side-chain lysine residue or N-terminal amine group may be prepared by standard solid phase peptide synthesis techniques, or by standard solution phase peptide synthesis techniques known to those skilled in the art.

Alternatively, compounds of general formula (XVIa) may be prepared from an active moiety, such as a glycosylamine for example by chemoselective nucleophilic substitution of the amine by the dihydrazide compound (XVIIIa):
H₂NNHCO(CH₂)ₜCONHNH₂ (XVIIIa)

Compounds of general formula (XVIb) may be prepared in an equivalent manner from an active moiety, such as a glycosylamine for example by chemoselective nucleophilic substitution of the amine by the carbonyl dihydrazide compound (XVIb):
H₂NNHCONHNH₂ (XVIIIb)

Compounds of general formula (XVIc) can be prepared from an active moiety by many methods known in the art towards the introduction of a carbonylhydrazide into an organic molecule.

In compounds of formula (XVa-e), (XVIa) and (XVIIa) and (XVIIIa), the group —(CH₂)ₜ— is preferred, but may be replaced by C₁₋₇ alkyl, C₃₋₁₀ cycloalkyl or Ar—C₀₋₇ alkyl group.

The technology described herein with particular exemplification in the controlled conjugation of active moieties to carriers, has many applications, both in solution and on a solid phase support. In particular, compounds of general formula (XV) are of use in medical applications and therefore the invention further provides a compound of general formula (XV) for use in medicine. The use in medicine may be either for a therapeutic or a diagnostic purpose. Compounds of general formula (XV) in which the active moiety E or G is a therapeutic agent may be used in the treatment of an appropriate medical condition. Alternatively, when E or G is an antigen, the compound of general formula (XV) may be useful as a vaccine. The compounds of general formula (XV) may also be used in various diagnostic applications, for example in solid or solution phase assays.

Examples of such applications include but are not restricted to the following.

Solution Phase Applications

The chemical linkage of a carrier (examples of which include, but are not restricted to, peptides, proteins, sugars, lipids, nucleic acids etc.) to a ligand (examples of which include, but are not restricted to, peptides, proteins, sugars, lipids, nucleic acids, alkaloids, vitamins, small organic molecules etc.) using the composition of this invention.

Therefore, in another aspect of the present invention there is provided a compound of general formula (XV) which is soluble in aqueous solution.

Solution phase applications of this invention include, but are not restricted to the following.

Conjugation of Epitopes/Mimotopes to Carriers Such as Proteins.

(See EXAMPLES 1-5)

When the active moiety is an epitope or mimotope, it may be a fragment, for example an antigenic determinant, derived from a protein or peptide molecule or a variant or analogue of such a molecule or a carbohydrate e.g. a surface oligosaccharide derived from a pathogenic organism such as a bacteria. Examples of epitopes and mimotopes which can be used with this method include oxytocin and analogues thereof. The carrier will, in many cases, be a protein.

The present invention enables higher epitope/mimotope concentrations to be loaded onto the carriers with the retention of epitope/mimotope-carrier conjugate solubility, thus improving the immune response. Since the conjugation is a controlled process, more than one agent may be conjugated to the carrier, allowing carriage of single and multiple immunologically relevant epitopes/mimotopes (e.g. B-cell and T-cell epitopes/mimotopes). Conjugation of epitopes/mimotopes may also be combined with co-conjugation of immunomodulating compounds (e.g. lipids, adjuvants, immunostimulating DNA sequences, cytokines, etc.).

Therefore, in a further aspect of the invention, there is provided a compound of general formula (XV) in which E or G is derived from an epitope or mimotope.

Optionally, the compound of general formula (XV) includes another active moiety, for example an immunomodulating compound such as a lipid, adjuvant, immunostimulating DNA sequences or cytokine attached to the carrier.

Compounds of general formula (XV) in which E or G is derived from an epitope or mimotope, can be used in a method for raising specific antibodies against the epitope or mimotope, the method comprising immunising a subject with a compound of general formula (XV).

Thus, the invention also provides a compound of general formula (XV) in which E or G is derived from an epitope or mimotope for immunising a subject in order to raise antibodies to the epitope or mimotope and the use of a compound of general formula (XV) in the preparation of an agent for raising antibodies against the epitope or mimotope.

Immunogenic compounds of general formula (XV) are of use as vaccines and therefore, in a further aspect of the invention there is provided a compound of general formula (XV) in which E or G is derived from an epitope or mimotope for use as a vaccine and also a pharmaceutical composition comprising a compound of general formula (XV) in which E or G is derived from an epitope or mimotope together with a pharmaceutically acceptable excipient.

The pharmaceutical composition may be a vaccine composition, in which case it may also comprise a pharmaceutically acceptable adjuvant.

Spectrophotometric Characterisation of the Reaction of a Linker of General Formula (I) (e.gTML (14)) with Proteins

(See EXAMPLE 6)

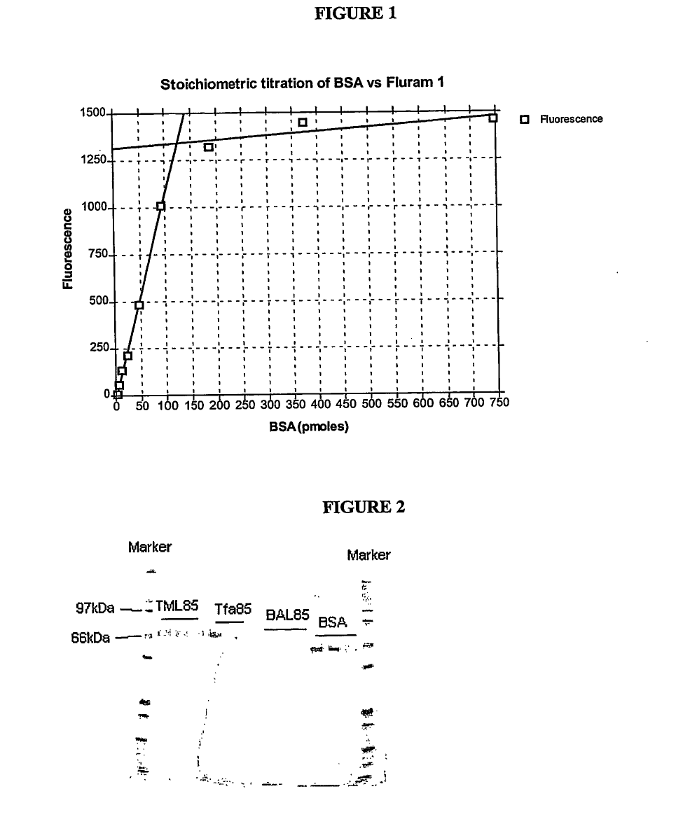

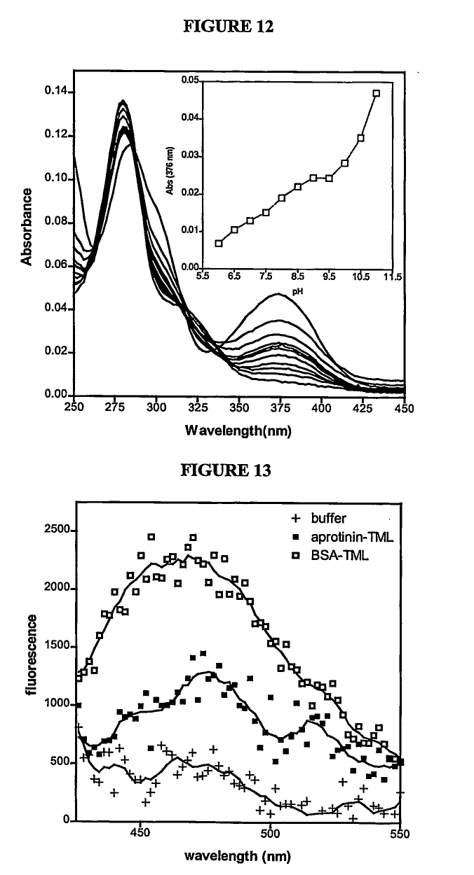

The determination of protein concentration has routinely been carried out by indirect and/or direct methods. The simplest indirect methods rely upon the reaction of proteins with chromogenic (Gornall, et. al., (1949), J. Biol. Chem., 147, 751; Lowry, et. al., (1951), J. Biol. Chem., 193, 265; Bradford, (1976), Anal. Biochem., 248, 72; Smith, et. al., (1985), Anal. Biochem., 150, 76) or fluorogenic reagents (Haugland, R. P., (2002), Handbook of fluorescent probes and research chemicals, Molecular Probes, Inc., Eugene, Oreg., USA). Practically, this has usually been carried out by either reacting proteins with dye reagents that display chromogenic characteristics upon binding to the protein or by derivatisation of the proteins with specific reagents. Indirect methods are usually destructive in nature and protein samples are not easily recoverable. Direct methods on the other hand rely on the measurement of absorption spectra due to the presence of specific amino acids, usually phenylalanine, tyrosine and/or tryptophan, and/or the peptide bond. This method is relatively simple and non-destructive allowing the proteins sample to be recovered. Due to the nature of naturally occurring amino acids, either as monomers (amino acids) or polymers (peptide or proteins), their absorption spectra, in the absence of any prosthetic group, is limited to wavelengths below 300 nm (Teale and Weber, (1957), Biochem. J. 65, 476-482). This means that protein spectral characterisation is routinely carried out at wavelengths below 300 nm and this makes it possible to monitor the reaction of a protein carrier with a linker of general formula (I). At or below neutral pH, linkers of general formula (I) in which R₂ is H and X is O (for example the TML linker (14)) have minimal absorption above 300 nm; however at pH values greater than neutral such linkers exhibit hyperchromic spectral characteristics due to ionisation of the hydroxyl functionality to the phenoxide species. These hyperchromic shifts are retained when the linker of general formula (I) has been reacted with proteins (for example the reaction of the TML linker with BSA giving BSA-TML, an example of a compound of general formula (XIVa)). This spectral property together with the negligible absorption of apo-proteins above 300 nm, enables the extent of the linker reaction (i.e. linker-loading) of proteins to be monitored and quantified directly from the absorption spectra (see FIG. 12 for spectral assessment of the BSA-TML species), for example at a wavelength of 376 nm. This offers a clear advance when compared to the currently used methods for assessing the extent of protein derivitisation such as the use of the fluorogenic reagent Fluram (see Example 2 and FIG. 1). The absorbance method is simple and non-destructive whereas the Fluram method requires experimental methodology and is a destructive technique.

Additionally, upon excitation at a wavelength above 300 nm (for example about 375 mm), proteins derivatised with linkers of general formula (I) wherein R₂═H, X=oxygen (e.g. BSA-TML) exhibit fluorescence emission (see FIG. 13). The progress of the reaction between the linker and the protein can therefore be monitored by fluorescence spectroscopy.

In addition to the extent of final derivatisation, the rate of derivatisation of a protein, for example BSA, with linker of general formula (I) wherein R₂═H, X=oxygen, for example TML linker (14), may be assessed in a non-destructive manner by measurement of absorbance or fluorescence spectra Following initiation of the coupling reaction, analytical samples may be removed, rapidly processed to isolate the linker-protein species from unreacted linker (process methods are available to perform such isolations experimentally within minutes). The isolated linker-protein species may then be quantified from the absorption spectral measurement (concentrations may be calculated from a calibration graph utilising Beer-Lamberts law; Atkins, (1984), Physical Chemistry, Second Ed., Oxford University Press, Oxford, UK) or fluorescence spectral measurement. If desired, the analytical linker-protein sample may then be returned to the bulk reaction.

Therefore, in a further aspect of the invention there is provided a non-destructive method of quantifying the extent and/or rate of reaction of a protein with a linker of general formula (I) in which R₂ is H and X is O, the method comprising either:
a) measuring the intensity of the absorbance spectrum at a wavelength above 300 nm and at a pH greater than 7 in order to detect the formation of a compound of general formula (XIV) in which R₂ is H and X is O; or
b) measuring the fluorescence emission upon excitation at a selected wavelength in order to detect the formation of a compound of general formula (XIV) in which R₂ is H and X is O.

When the process is used for the measurement of the rate of reaction, it will include a plurality of measuring steps so that the variation in the intensity of the absorbance spectrum or of the fluorescence emission over time can be calculated in order to determine the rate of product formation.

The process is typically carried out at room temperature (about 18 to 25° C.) and at a pH of about 7 to 11 and more usually pH 7-9.5.

The absorption spectrum may be measured at a wavelength between 300 and 400 nm, typically about 350-400 nm.

A typical suitable wavelength for excitation in order to measure the fluorescence emission is 300-400 nm, preferably about 375 nm.

Analytical Assessment of Chemoselective Addition of Ligands to Linker-Proteins (XIV) to Provide Protein Constructs (XV).

(See EXAMPLES 7 and 8)

The linkers of the present invention have the ability to react chemoselectively with a diverse set of proteins, from a range of sources (e.g. viral, bacterial, mammalian, etc.) with a broad range of molecular weights (˜6.5 kDa to 205 kDa) providing compounds of general formula (XIVa).

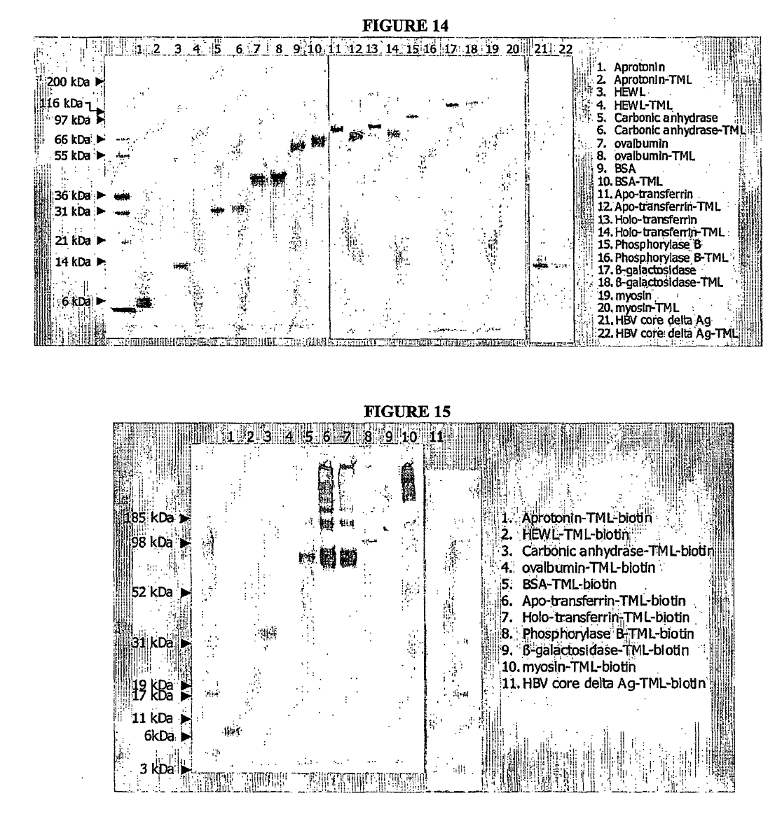

Examples of proteins which can be labelled with the charged-balanced linker of general formula (I) (for example the TML linker (14)), through the use of a carboxyl activated analogue such as compound (15) cover an extensive molecular weight range. Included are aprotonin (6.5 kDa), hen egg-white lysozyme (14 kDa), hepatitis B virus core delta antigen (17 kDa), carbonic anhydrase (29 kDa), ovalbumin (45 kDa), bovine serum albumin (66 kDa), apo-transferrin (88 kDa); holo-transferrin (88 kDa); phosphorylase B (97.4 kDa), β-galactosidase (116 kDa) and myosin (205 kDa). Each of the compounds of general formula (XIV) may undergo further chemoselective elaboration with a set of hydrazides, including, but not limited, to biotin-hydrazide, Texas Red-hydrazide and oxytocin-hydrazide to provide compounds of general formulae (XVa) in which R₁₃ is (CH₂)ₜCONHE or G. These applications are demonstrated in FIGS. 14 and 15 which illustrate the gel-shift analysis of proteins upon chemoselective reaction with activated TML linker (15) and then subsequent derivatisation with biotin hydrazide to provide protein constructs analysed by Western blot analysis for the presence of biotin.

Spectrophotometric Characterisation of Reaction of TML Linked Proteins with Biotin-Hydrazide to Provide Biotin Labelled Protein Constructs

(See EXAMPLES 7 and 8)

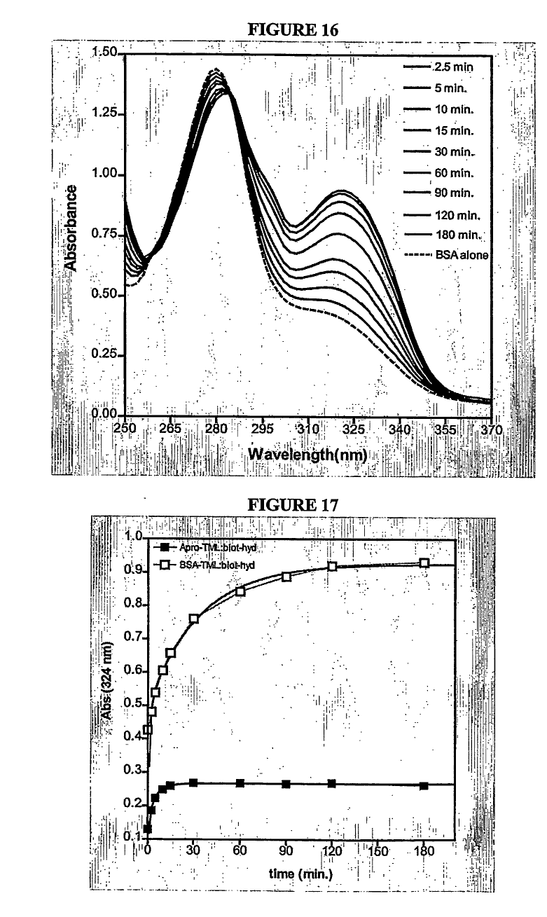

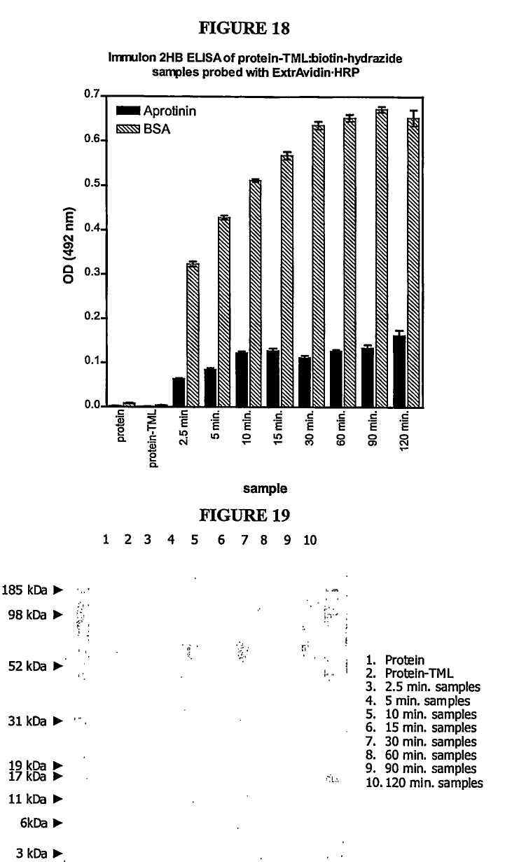

As detailed above, the reaction of a protein with a linker of general formula (1) (wherein R₂═H, X=oxygen) provides a linker-protein species that exhibits an absorbance maximum at a wavelength higher than 300 nm, for example 376 nm when assessed at pH values above neutral (where the phenoxide ion exists) (see FIG. 12). The absorbance characteristics of this species change dramatically when assessed at acidic pH values, for example pH 3.5, where little absorbance above 300 nm is observed (see FIG. 16,—BSA alone). Reaction of a protein-linker species of general formula (XIVa), for example protein-TML, with a hydrazide of general formula (XVIa, b or c), for example biotin-hydrazide, provides the active moiety linked protein construct of general formula (XVa), for example BSA-TML-biotin or aprotinin-TML-biotin. Now when assessed at pH 3.5, the constructs of general formula (XVa) exhibit a hyperchromic shift that is proportional to the extent of reaction between protein-linker of general formula (XIVa) and the hydrazide of general formula (XVIa, b or c). This spectral property, which arises due to an extension of the aromatic chromophore upon conjugation with the unsaturated hydrazone bond, enables the rate and extent of the reaction of protein-linker species with hydrazide to be monitored and quantified directly from the absorption spectra (see FIGS. 16 and 17). The absorbance measurement is a quantitative assessment of loading, and, as an example of this, FIG. 17 shows the total absorbance measured at 324 nm, pH 3.5 for the formation of the BSA-TML-biotin and aprotinin-TML-biotin constructs of general formula (XV). The total concentration of surface lysine residues available for reaction with a linker of general formula (I) is approximately 4 times that of aprotinin for BSA. This ratio is clearly reflected in the absorbance maximum of 0.25 (aprotinin) compared to 0.90 (BSA). The reaction may also be followed by ELISA analysis (FIG. 18) and Western blot analysis (FIG. 19).

The combined ability to control both the chemoselective nature of construct formation along with a real-time quantitative analysis of the extent of construct formation provides clear and significant advances upon the current methods used for production of ligand linked proteins.

Therefore, in yet another aspect of the invention there is provided a non-destructive method for quantifying the extent and/or rate of reaction of a linker-protein of general formula (XI) wherein R₂ is H and X is O, with an active moiety hydrazide, the process comprising measuring the intensity of the absorbance spectrum at a wavelength above 300 nm and a pH less than 7.

When the process is used for the measurement of the rate of reaction, it will include a plurality of measuring steps so that the variation in the intensity of the absorbance spectrum over time can be calculated in order to determine the rate of product formation.

The process is typically carried out at room temperature (about 18 to 25° C.) and at a pH of about 2 to 6, more usually pH 3-5 and preferably pH 3-4.

A typical wavelength at which the absorbance spectrum may be measured is 300-400 nm.

Using this process, it is possible to prepare a calibration graph or table and to calculate the maximum possible absorbance intensity for different carriers. The maximum absorbance intensity for a carrier is related to the number of available Q residues on that carrier.

Since the formation of the ligand derivatised linker-protein construct can be monitored in real-time by assessing the absorbance signal at above 300 nm (e.g. 324 nm in the case of a protein-TML-biotin construct), the controlled addition of multiple different active moiety hydrazides may be achieved by sequential addition of single active moiety hydrazides for set reaction times. For example, ligand hydrazide 1 may be added to a compound of general formula (XIV) e.g. BSA-TML until the absorbance measurement at 324 nm, pH 3.5 has reached 50% of the maximum value, then the reaction medium changed to ligand hydrazide 2. Continuation of reaction until the absorbance measurement at 324 nm, pH 3.5 has reached 100% of the maximum value provides a ligand-linker-protein construct of general formula (XV) that contains a 50% loading of each of ligand hydrazides 1 and 2. For example, a single carrier protein could be derivatised with both a B-cell and T-cell antigen to provide a construct with improved immunogenic and antigenic responses.

In this way, it is possible to prepare a compound of general formula (XV) which has a selected proportion of its available residues loaded with an active moiety or, alternatively, which has selected proportions of its available residues Q loaded with two or more different active moieties.

Thus, in a further aspect of the invention, there is provided a process for the preparation of a compound of general formula (XV) as defined above in which:
R₂ is H and X is O;
the carrier has multiple residues Q;
a first selected percentage of the Q residues is derivatised with a first active moiety; and, optionally
further selected percentages of the Q residues are derivatised with further active moieties;
   the process comprising:
a. reacting a compound of general formula (XIV) in which R₂ is H and X is O with a first compound of general formula (XVI) at a pH less than 7;
b. monitoring the progress of the reaction by measuring the intensity of the absorbance spectrum at a wavelength of above 300 nm and stopping the reaction when the intensity of the absorbance spectrum reaches the first selected percentage of the known maximum intensity; and optionally
c. reacting the product of steps (a) and (b) with one or more further compounds of general formula (XVI), monitoring the progress of the reaction by measuring the intensity of the absorbance spectrum at a wavelength of above 300 nm and stopping the reaction when the intensity of the absorbance spectrum reaches further selected percentages of the known maximum intensity.

As with the process for quantifying the extent or rate of reaction of a linker-protein with a hydrazide, this process is typically carried out at room temperature (about 18 to 25° C.) and at a pH of about 2 to 6, more usually pH 3-5 and preferably pH 3-4.

A typical wavelength at which the absorbance spectrum may be measured is 300-400 nm.

An alternative method of achieving a compound of general formula (XV) loaded with different active moieties in selected proportion is the use of an isokinetic mixture of active moiety-hydrazides (i.e. a mixture that is biased in molar terms to compensate for the differing rates of reaction for different hydrazides). It is possible to calculate the correct proportions of the isokinetic mixture if the rates of reaction of different active moieties with the compound of general formula (XIV) and the maximum loading of the carrier have been calculated using the methods described above.

Simple replacement of the experimentally exemplified active moieties and proteins of the invention show that the same basic principals can be used to monitor the reaction of any active moiety hydrazide with any protein through the use of a linker of general formula (I) (wherein R₂═H, X=oxygen).

Characterisation of the Cleavage Reaction of Protein-Linker-Active Moiety Constructs.

(See EXAMPLE 9)

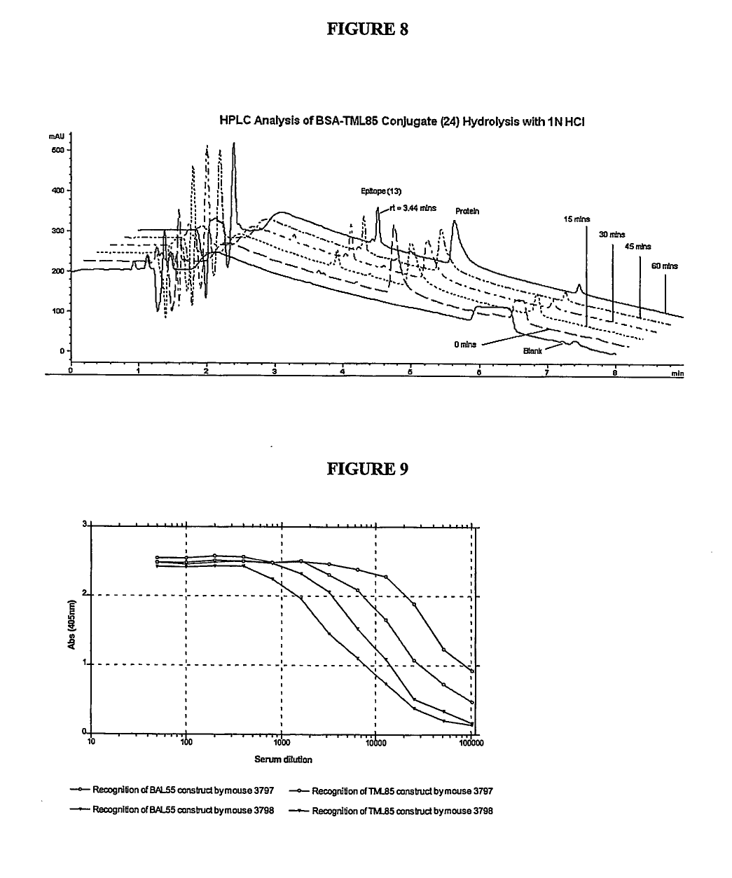

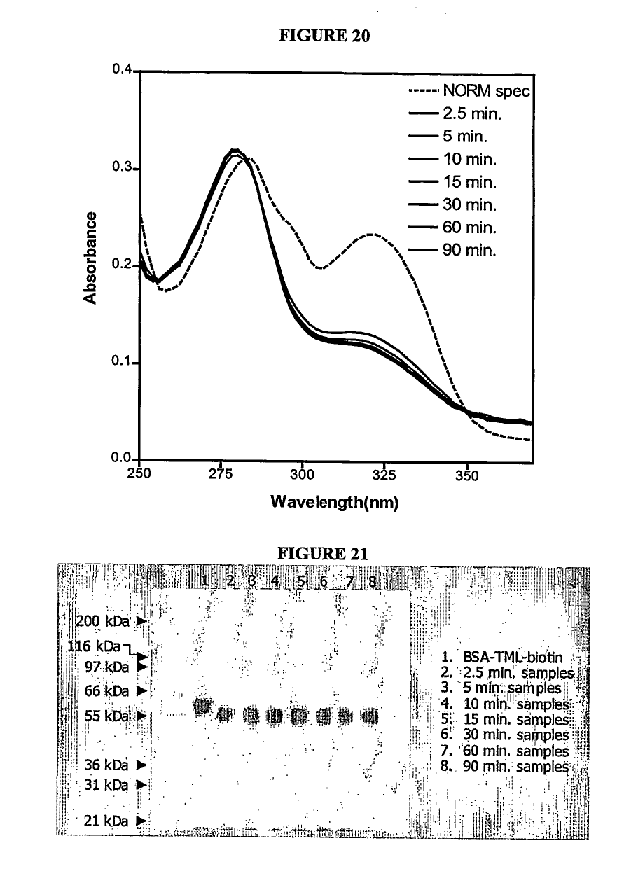

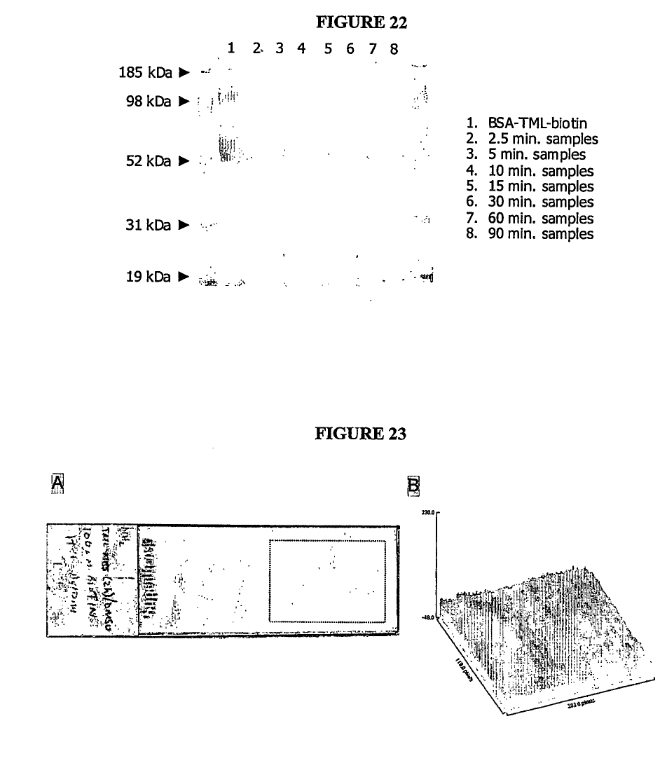

FIG. 8 details the qualitative and quantitative cleavage of an example compound of general formula (XV) to provide a compound of general formula (XIV) and the liberated active moiety hydrazide (in the example shown, the liberation of oxytocin epitope (13)). The cleavage reaction, and hence analytical assessment of the loaded active moiety, may also be monitored by a reverse of the absorbance characteristics described above which were used to monitor the loading reaction. Therefore, the real time monitoring of release of a active moiety hydrazide from a compound of general formula (XV) may be accomplished by assessment of the reduction of the absorbance spectral peak at a wavelength above 300 nm (for example 324 nm in Example 9 and FIG. 20), upon treatment of protein-linker-active moiety construct with an acid, e.g. 1N HCl (see FIGS. 20, 21 and 22). FIG. 20 shows a clear reduction in the 324 nm absorbance as cleavage time progresses (opposite of the effect detailed in FIG. 16), whilst FIG. 22 shows a parallel reduction in the intensity of the Western blot stain to biotin of the cleavage constructs.

In a further aspect of the invention there is provided a method for quantifying the extent and/or rate of release of an active moiety hydrazide from a compound of general formula (XV) in which R₂ is H and X is O, the method comprising the measurement of the absorbance spectrum maximum at a wavelength above 300 nm and at pH less than 7.

When the process is used to calculate the rate of release of the active moiety hydrazide from the construct, a plurality of measurements will be required in order to calculate the reduction over time in the absorbance spectrum intensity.

Again, this process is typically carried out at room temperature (about 18 to 25° C.) and at a pH of less than 3 and more usually less than pH 2.

A typical wavelength at which the absorbance spectrum may be measured is 300-400 nm.

Solution Phase Biochemical/Biophysical/Biomedical Applications.

EXAMPLES 7, 8 and 9 have detailed that essentially any hydrazide functionalised active moiety, be it an epitope, mimotope or a ligand such as a small molecule drug, new chemical entity (NCE) or diagnostic marker, can be chemoselectively linked in a controlled manner to a whole host of proteins and furthermore cleaved in a quantified and controlled manner. This opens up an extensive range of screening and diagnostic applications.

For example, chemical linkage of a ligand to a carrier to enable molecular interactions to be monitored. In this case the definition of ligand may be extended to include labelling moieties such as chromophores (biochemical, biophysical or chemical), fluorophores (biochemical, biophysical or chemical), luminophores (biochemical, biophysical or chemical), phosphorescence, radiochemicals, quantum dots, electron spin tags, magnetic particles, nuclear magnetic resonance tags, x-ray tags, microwave tags, electrochemical, electrophysical (e.g. increased resistance), surface plasmon resonance, calorimetry, etc. Using the present invention carriers would be tagged by a ligand, creating a soluble intermediate with which molecular interactions could be monitored by a complementary physical, chemical or biological technique.

Therefore, in a further aspect of the invention, there is provided a compound of general formula (XV) in which E or G is a labelling moiety.

Specific examples of labelling moieties include biotin, and chromophores such as Texas Red®.

The principles of example diagnostic applications are detailed in FIGS. 18 and 19. Here, the formation of the BSA-TML-biotin and aprotinin-TML-biotin constructs of general formula (XV) has been characterised by an ELISA (FIG. 18) and Western blot for biotin analysis (FIG. 19). For the ELISA assays, the protein-TML-biotin samples (a time-course experiment monitoring formation of the construct) were absorbed onto an Immulon 2HB microtitre plate and probed with a labelled antibody to biotin. FIG. 18 clearly shows the timecourse of construct formation and closely complements the data detailed for the identical reaction monitored alternatively by absorbance measurements shown in FIGS. 16 and 17. A Western blot for biotin analysis (FIG. 19) (a time-course experiment monitoring formation of the construct) clearly shows the timecourse of construct formation and closely complements the data detailed for the identical reaction monitored alternatively by absorbance measurements and ELISA analysis shown in FIGS. 16, 17 and 18.

A typical practical diagnostic application may be founded based upon the principles detailed above as follows. One or more known pathogenic antigen(s) may be chemoselectively coupled to a carrier protein through the use of a linker of general formula (I), exploiting the quantitative and qualitative benefits detailed above. The protein-linker-antigen(s) construct may then be absorbed onto a surface (e.g. a diagnostic strip) and the surface contacted with a biological sample of a patient that is suspected of having an illness caused by the pathogen(s) from which the antigen(s) are derived. If the patient has the pathogenic illness, an antibody response will bind to the protein-linker-antigen(s) construct on the surface. The surface may then be probed with a general labelled anti-antibody to generate a qualitative response, which, if present is an indication that the patient has the pathogenic illness.

Solid Phase Applications

The chemical linkage of a solid phase (i.e. non-solution phase) examples of which include, but are not restricted to, synthetic materials (such as hydrocarbon-based plastics, polymers, glass, gels, resins, etc.), natural polymers such as proteins, sugars (e.g. cotton), lipids (liposomes), etc. to a ligand (examples of which include, but are not restricted to, peptides, proteins, sugars, lipids, nucleic acids, alkaloids, vitamins, etc.) using the composition of this invention.

Therefore, in a further aspect of the invention there is provided a compound of general formula (XV) where the carrier is a solid surface that is insoluble in aqueous solution.

Solid phase applications of the present invention include, but are not restricted to those set out below.

Solid Phase Biochemical/Biophysical Applications.

(See EXAMPLE 10)

Techniques in which a reagent, or reagents, of choice may be immobilised on a surface (i.e. solid phase) enables exposure of the immobilised reagent to a wide range of solutes which, after incubation, may be removed by washing. Immobilisation therefore allows retention of the reagent while solutes are interchangeable. This technique therefore enables multiple steps to be performed on a single reagent without loss of reagent and allows more specific detection by removal of unwanted solutes. Techniques which employ such a methodology include, but are not limited to, chemical linkage of a ligand and/or carrier to a solid phase to enable molecular interactions to be monitored. In this case the present invention could be used for applications such as enzyme linked immunosorbent assays (ELISAs), surface plasmon resonance, quartz crystal microbalances, atomic force microscopes, etc. Selective covalent linkage of material to solid surfaces, will also allow generation of microarrays (including but not limited to peptides, proteins and nucleic acids) (e.g. see FIG. 23 for the attachment and release of biotin hydrazide to a glass plate).

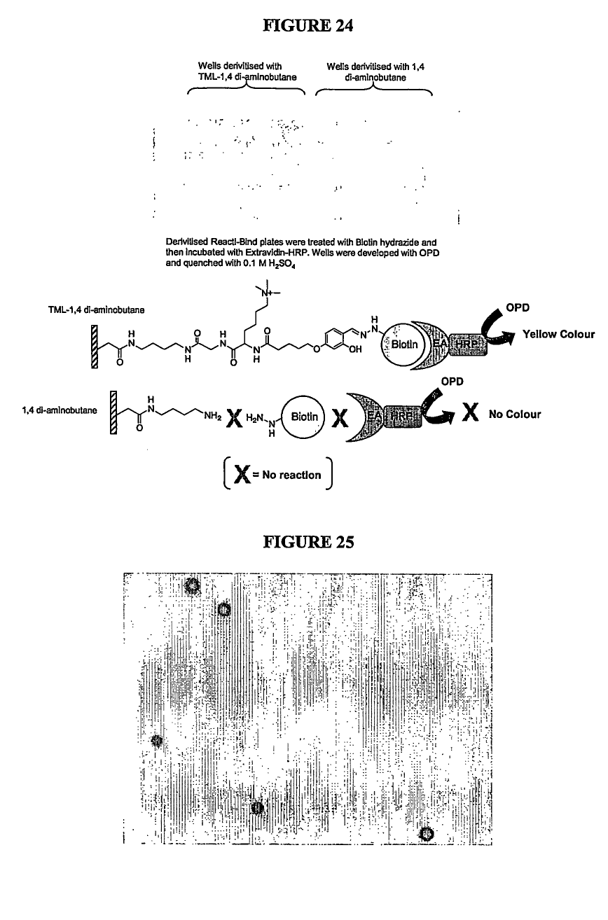

In addition to the quantitative and qualitative applications detailed above, this controlled release reaction has other applications where a capture and release mechanism is useful. For instance, a compound of general formula (XV) such as a protein-linker-ligand may be immobilised onto a 96-well plate (e.g. see FIG. 24 for the attachment and ELISA analysis of biotin hydrazide to a 96-well plate).

Separation/Purification.

(see EXAMPLE 11)

Purification is a basic technique utilised in the life sciences (Scopes, R. K., (1993), Protein Purification: Principles and Practice, 3^{rd} Ed., Springer-Verlag New York, Incorporated; Williams, B. L. and Wilson, K., (1983), A Biologist's guide to Principles and techniques of Practical Biochemistry, 2^{nd} Ed., Edward Arnold (Publishers) Ltd., London). Many different methods are used for purification of a wide range of molecules, which are separated from mixtures in order to produce purified materials for study. An example of purification is chromatography, whereby molecules are separated on the basis of physiochemical properties upon partitioning of molecules between a solid phase (i.e. resins) and a solution phase. Chromatography may be categorised into various techniques based upon the resin and the solution phases employed. Examples include, but are not limited to, ion-exchange, reverse-phase, gel filtration, hydrophobic, chromatofocusing, affinity, etc. (Scopes, R. K., (1993), Protein Purification: Principles and Practice, 3^{rd} Ed., Springer-Verlag New York, Incorporated; Williams, B. L. and Wilson, K., (1983), A Biologist's guide to Principles and techniques of Practical Biochemistry, 2^{nd} Ed., Edward Arnold (Publishers) Ltd., London).

An example of the use of a linker of general formula (I) in the purification (i.e. capture) of a protein from a mixture is detailed in FIG. 24. A sepharose bead is derivatised with TML linker (14) to give a compound of general formula (XIVa) which is then reacted with biotin hydrazide to give a compound of general formula (XVa) wherein the ‘carrier’ is a sepharose bead. This biotin derivatised bead is then used to attract (capture) the protein ExtrAvidin-HRP from solution. Addition of a substrate that develops a colour in the presence of ExtrAvidin-HRP confirms the presence of ExtrAvidin-HRP by colour staining of the sepharose bead (FIG. 24).

Chemical linkage of a ligand to a solid phase enables selective separation of molecules based on physicochemical properties. Applications include, but are not restricted to, affinity purification, chiral separation, etc.

When the compounds of general formula (XV) are for use in assay or separation/purification methods, E and G will often be derived from a ligand which is specific for the analyte or a compound to be separated. An additional active moiety E or G, such as a labelling molecule may also be bound to the carrier.

The invention thus provides a method of separating a compound from a mixture, the method comprising contacting the mixture with a compound of general formula (XV) as described above in which E or G is a ligand which binds specifically to the compound to be separated and the carrier is a solid support.

The invention also provides an assay method comprising contacting a mixture suspected of containing an analyte with a compound of general formula (XV) as described above in which E or G is a ligand which binds specifically to the analyte and the carrier is a solid support.

Medical Devices.

Chemical derivatisation of medical devices and consumables allowing presentation of biologically active or inert molecules at a tissue/solid-surface interface. For example controlled conjugation of peptide growth factors, chemo-attractant proteins or analogues of both, to functionalised polymers commonly used in modern coverings, may allow development of next generation, bioactive wound dressings. In a further example, dialysis tubing may be derivatised with the linker in order to allow heparin to be coupled onto the surface of the polymer, decreasing the risk of contact activation of the blood coagulation process.

The invention also provides a wound dressing comprising a compound of general formula (XV) wherein the carrier is a functionalised polymer of the type commonly used in wound dressings and E or G is a peptide growth factor, a chemo-attractant protein, a ligand or an analogue of one of these.

The invention also provides a method of treating wounds comprising applying to the wound a dressing as described above. Also provided by the invention are (1) a compound of general formula (XV) wherein the carrier is a functionalised polymer of the type commonly used in wound dressings and E or G is a peptide growth factor, a chemo-attractant protein, a ligand or an analogue of one of these for use in the treatment of wounds; and (2) the use of such a compound in the preparation of a dressing for use in the treatment of wounds.

In still another aspect of the invention, there is provided dialysis tubing comprising an insoluble compound of general formula (XV) wherein the carrier is a polymer suitable for use in dialysis tubing and E or G is heparin.

Furthermore, there is provided a compound of general formula (XV) wherein the carrier is a polymer suitable for use in dialysis tubing and E or G is heparin for use in the preparation of dialysis tubing.

The invention will now be discussed in greater detail with reference to the drawings described below and the Examples, which are not intended to be limiting.

FIG. 1 shows the stoichiometric titration of BSA against the amine specific fluorescent reagent FLURAM 1™. By keeping one reactant constant and gradually increasing the other, a plateau was reached, indicating a point of equivalence. Since the number of free amines in BSA is known, an estimate of the number taking part in the reaction was made (21-25).

FIG. 2 is an sodium dodecyl sulphate-polyacrylamide electrophoresis gel showing the molecular weight of BSA compared with that of three BSA constructs, TML85, Tfa85 and BAL85.

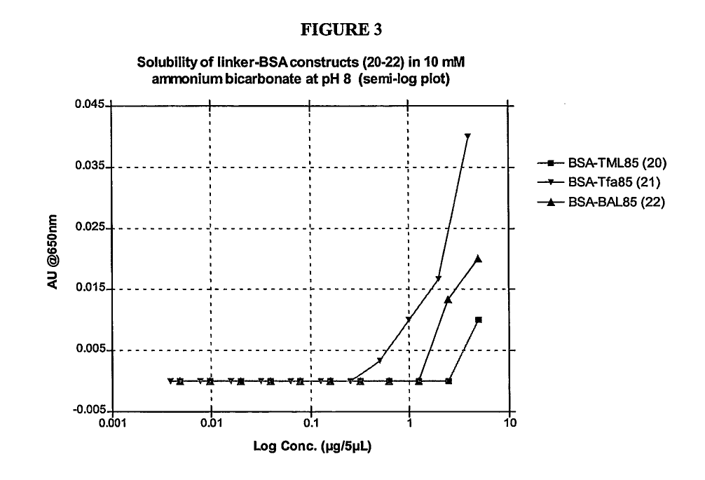

FIG. 3 is plot of absorbance units (AU) at 650 nm vs. log concentration and illustrates the solubility of linker BSA constructs (20-22) in 10 nM ammonium bicarbonate at pH 8.

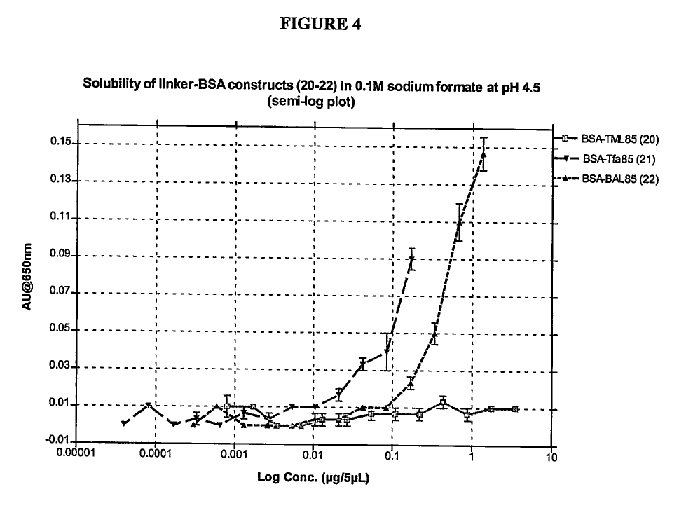

FIG. 4 is a plot of absorbance units (AU) at 650 nm vs. log concentration and illustrates the solubility of linker-BSA constructs (20-22) in 0.1M sodium formate at pH 4.5.

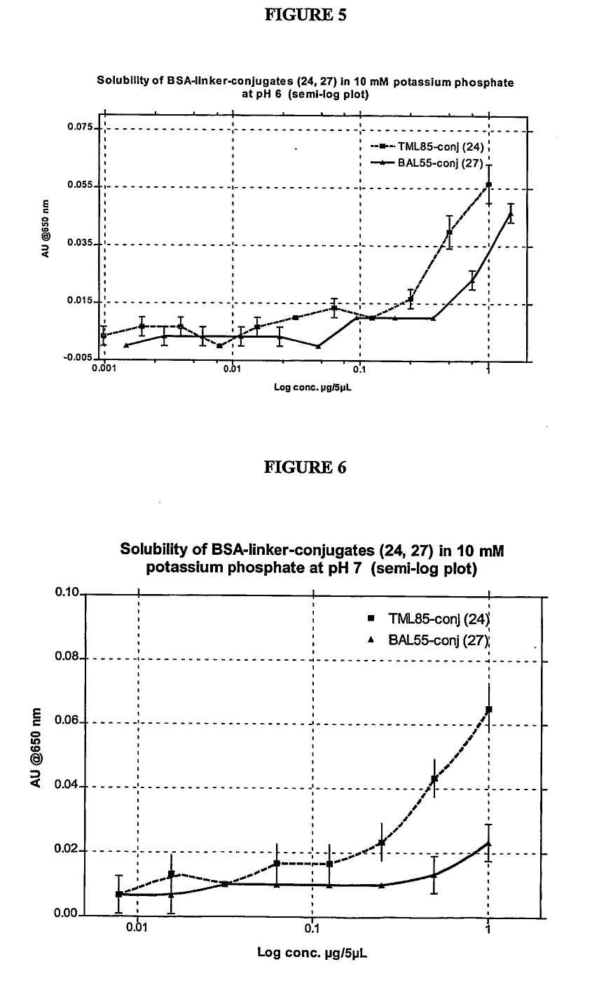

FIG. 5 is a plot of absorbance units (AU) at 650 nm vs. log concentration and illustrates the solubility of linker-BSA constructs (20-22) in 10 mM potassium phosphate at pH 6.

FIG. 6 is a plot of absorbance units (AU) at 650 nm vs. log concentration and illustrates the solubility of linker-BSA constructs (24 and 27) in 10 mM potassium phosphate at pH 7.

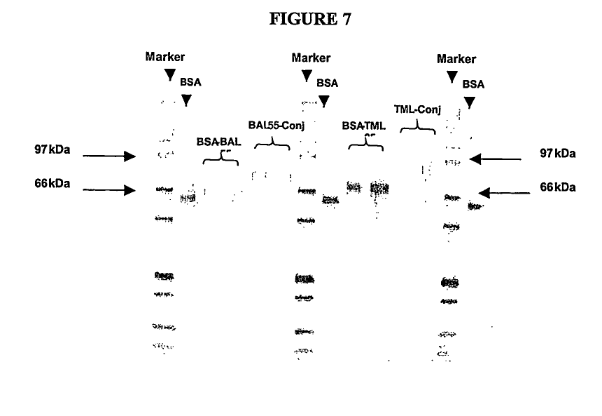

FIG. 7 is an sodium dodecyl sulphate-polyacrylamide electrophoresis gel showing the molecular weight of BSA compared with that of BSA.BAL, BAL55-Conj, BSA.TML and TML-conj.

FIG. 8 shows the HPLC analysis of the BSA-TML85-epitope conjugate (24) after hydrolysis with 1N hydrochloric acid and shows that hydrolysis regenerated BSA-TML85 and the epitope (13).

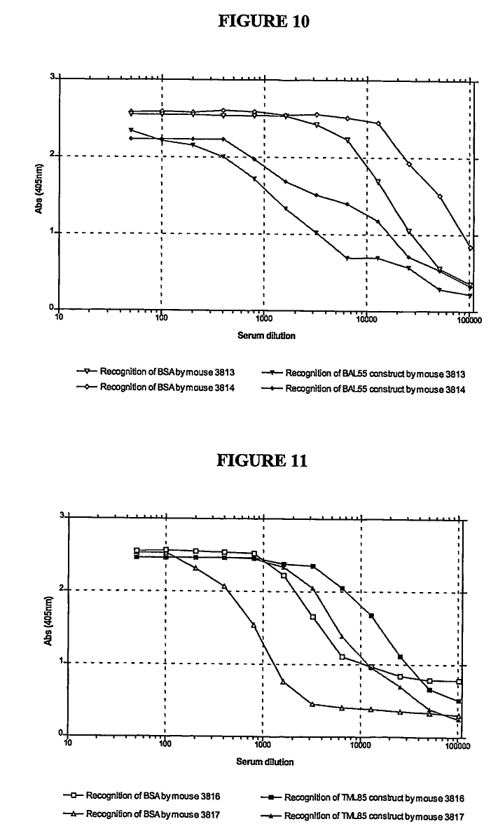

FIGS. 9 to 11 show the results of ELISA analysis of sera from mice immunised with BSA alone (FIG. 9) or with oxytocin conjugated to BSA using either BAL linker (FIG. 10) or TML linker (FIG. 11). Both constructs are recognised by antibodies raised to BSA alone, which is to be expected since BSA is the carrier protein present within both constructs and thus provides a positive control.

FIG. 9 shows that titres of antibodies that recognise both BSA-BAL55-oxytocin and BSA-TML-oxytocin constructs are raised in mice immunised with BSA alone.

FIG. 10 shows that titres of BSA (non-specific) and BSA-BAL55-oxytocin construct (specific) are raised in mice immunised with BSA-BAL55-oxytocin construct.

FIG. 11 shows that titres of BSA (non-specific) and BSA-TML85-oxytocin construct (specific) antibodies are raised in mice immunised with BSA-TML85-oxytocin construct.

FIG. 12 BSA-TML absorption spectra at various pH values exhibiting hyperchromic shift with increasing pH. Inset is a plot of the absorbance at 376 nm versus pH.

FIG. 13. Fluorescence emission spectra of diluted samples of post-dialysis buffer (100 mM sodium formate; pH 3.5); aprotinin-TML and BSA-TML at pH 12.

FIG. 14. Silver stained SDS-NuPAGE gel of proteins and TML-NHS (15) treated protein samples.

FIG. 15. TMB exposed-ExtrAvidinHRP treated PVDF blot of biotinylated protein samples.

FIG. 16. Hyperchromic change in BSA-TML absorption spectra upon addition of biotin-hydrazide at pH 3.5.

FIG. 17. Kinetics of absorbance change at 324 nm for BSA-TML (□) or Aprotinin-TML (▪) upon addition of biotin-hydrazide at pH 3.5.

FIG. 18. ELISA of samples from the kinetic reaction of Aprotnin-TML or BSA-TML upon addition of biotin-hydrazide (same samples as FIGS. 16 & 17).

FIG. 19. Western blot of quenched samples from the kinetic reaction of Aprotinin-TML or BSA-TML upon addition of biotin-hydrazide (same samples as FIGS. 16, 17, 18 & 22). The equivalent Aprotinin and BSA samples were pre-mixed prior to loading.

FIG. 20. Spectral changes in BSA-TML-biotin upon acidification to 1M HCl. Dotted line represents normalised spectra for un-treated BSA-TML-biotin produced from spectra for un-diluted sample.

FIG. 21. Nu-PAGE gel of samples from the kinetic reaction of Aprotinin-TML-biotin or BSA-TML-biotin upon acidification to 1 M HCl. Equivalent Aprotinin and BSA samples were pre-mixed prior to loading.

FIG. 22. Western blot of samples from the kinetic reaction of Aprotinin-TML-biotin or BSA-TML-biotin upon acidification to 1 M HCl. Equivalent Aprotinin and BSA samples were pre-mixed prior to loading.

FIG. 23. TMB developed-ExtrAvidinHRP treated glass slide elaborated with aminopropyl silane, TML linker and biotin-hydrazide. A, image of developed slide; B, three-dimensional surface intensity plot of a section of the slide (approx. area shown by dotted box).

FIG. 24. Row of a Reacti-Bind microtitre plate showing biotin hydrazide treated wells which have either been derivitised with TML-1,4-diaminobutane or 1,4-diaminobutane alone. Only the TML derivatised wells show binding of ExtrAvidin to biotin in the wells (producing a yellow colour).

FIG. 25. QX3 microscope captured image of TML-NHS and biotin-hydrazide treated EAH Sepharose beads exposed to ExtrAvidin-HRP and developed with TMB reagent. Control beads (ie. not treated with TML-NHS or biotin-hydrazide were colourless).

The experiments described below (EXAMPLES 1-5) exemplify the utilisation of a charge-balanced linker in the controlled conjugation of an epitope to bovine serum albumin (BSA) carrier protein. A series of in vitro solubility and in vivo immunisation experiments is described that clearly shows the superior characteristics of a charge-balanced linker construct for the generation of an antibody response to an immunogen. The exemplification is described as follows;
1. Synthesis of example epitope and linker structures
2. Solubility studies with BSA-linker constructs
3. Solubility studies with BSA-linker-epitope constructs
4. Chemical analysis of BSA-linker-epitope constructs
5. Immunisation studies with BSA-linker-epitope constructs
   Experimental Methods

All reagents were of the highest commercially available quality and were used as received. Unless otherwise stated all chemicals and biochemicals were purchased from the Sigma Chemical Company (Poole, Dorset, UK). All solid phase synthesis was performed using an “Fmoc/tBu” procedure, (see Atherton, E and Sheppard, R. C. in ‘Solid Phase Peptide Synthesis: A Practical Approach’, IRL Press, 1989.) Standard Fmoc amino acids were obtained from Chem-Impex International (Wood Dale, Ill., USA) and Novabiochem (Nottingham, UK) with the exception of Fmoc N-s-trimethyllysine, which was purchased from Bachem UK Ltd. (St. Helens, UK), along with Fluram (fluorescamine). PS-carbodiimide resin was obtained from Argonaut Technologies (Muttenz, Switzerland). All solvents were purchased from Romil (Cambridge, UK). Solid phase syntheses were performed manually in a polypropylene syringe fitted with a polypropylene frit to allow filtration under vacuum. Analytical HPLC was performed on Agilent 1100 series instruments including a G1311A quaternary pumping system, with a G1322A degassing module and a G1365B multiple wavelength UV-VIS detector. Data were collected and integrated with Chemstation 2D software. The analyses were performed on a Zorbax, 5 μm, C8 reverse phase column (150×4.6 mm i.d.), at a flow rate of 1.5 ml/min, monitoring at 215 and 254 nm. Eluents used were (A) 0.1% trifluoroacetic acid in water and (B) 90% acetonitrile/10% eluent A and used to run a gradient starting at 10% B, increasing to 90% B over 7 minutes, holding for 1 minute, returning to 10% B over 1 minute and then remaining at initial conditions for a further 4 minutes to allow column re-equilibration. Compounds were purified by semi-preparative HPLC, using a Phenomenex Jupiter C4 reverse phase column (250×10 mm i.d.) at a flow rate of 4 ml/min, using the equipment and eluents described above. The molecular weight of compounds was determined on an Agilent 1100 series LC/MSD electrospray mass spectrometer. BSA conjugates were concentrated using Centricon centrifugal filters (50,000 MWCO) (Millipore, Mass., USA) and purified by dialysis using Slide-A-Lyser dialysis cassettes (10,000 MWCO) (Pierce, Ill., USA). Molecular weight estimations and purity of the BSA conjugates were made by polyacrylamide gel electrophoresis in the presence. of sodium dodecyl sulfate (SDS-PAGE) using 4-20% NuPAGE gels (Invitrogen, Paisley, U.K.) employing the 3-(N-morpholino) propane sulfonic acid (MOPS) buffer system (Invitrogen) according the manufacturers instructions. Protein visualisation was carried out using the SilverExpress stain kit (Invitrogen). In all cases the gels were dried using the gel drying kit (Invitrogen) and for presentation purposes, gels were scanned at 300 dpi resolution using grey scale false colour (OfficeJet Pro1175c; Hewlett Packard). Fluram fluorescence assays were carried out in Microfluor W1 96-well microtitre plates (Dynex Thermo Lifesciences, UK) using a Gemini plate reader (Molecular Devices, Crawley, UK) and monitored at 390 nm (excitation) and 460 nm (emission). Turbidity measurements were made at 650 nm using a Spectramax384 96-well plate reader (Molecular Devices), carried out in 384-well PS microplates (Labsystems, Basingstoke, Hants, UK), while Bradford assays were measured at 595 nm in 96-well PS microplates (Greiner Bio-One Ltd., Stonehouse, Gloucestershire, UK).

Image capture, analysis and processing. Images were captured using a Hewlett Packard C7710A scanner employing HP Precision ScanPro 3.02 software on default settings. Images were routinely scanned at a minimum resolution of 600 d.p.i. using true color (32 bit). Images were marked with legends employing Powerpoint (Microsoft Corp.). Image analysis and processing was carried out using ImageJ software (http://rsb.info.nih.gov/ij/).

### EXAMPLE 1

### Synthesis of Example Epitope and Linker Structures

Synthesis of the oxytocin analogue (13) and linkers (14-19) proceeded smoothly using standard solution chemistries and Fmoc solid phase techniques (see Atherton, E and Sheppard, R. C. in ‘Solid Phase Peptide Synthesis: A Practical Approach’, IRL Press, 1989) to provide the desired compounds in good yield and purity. The linkers were stored as the free acids (14, 16, 18) and the activated succinimide ester of the linkers (15, 17, 19) were freshly prepared when required.

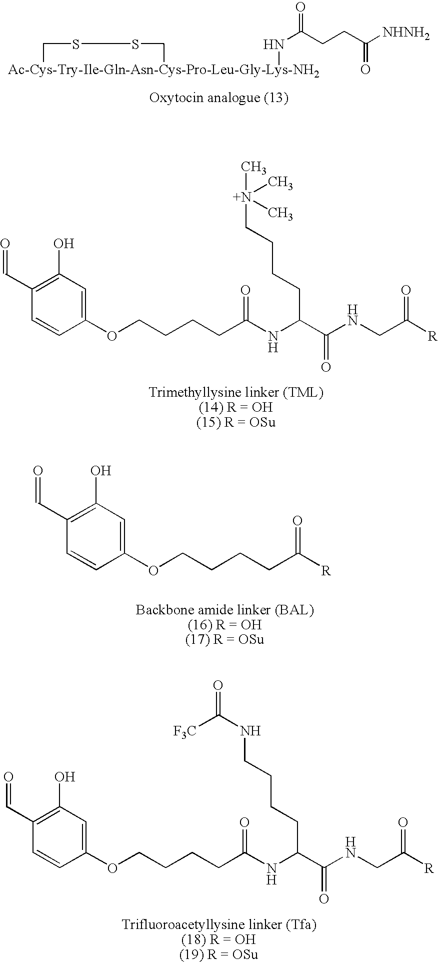

### A. Synthesis of Oxytocin Analogue (13)

Oxytocin analogue (13) with the sequence (one letter code) Acetyl-CYIQNCPLGK(COCH₂CH₂CONHNH₂)—NH₂, was synthesised manually using Fmoc/tBu protection strategy on TGR resin (0.25 g, 0.05 mmol, substitution: 0.2 mmol/g). Coupling of the Fmoc amino acids was accomplished with an HBTU/HOBt method utilising dimethylformamide as the solvent, using 3 equivalents of amino acid and coupling reagents with respect to the loading of the resin. The Fmoc group was removed by a 15 min treatment with 20% piperidine in dimethylformamide. The C-terminal lysine residue was introduced with Dde side chain protection, to allow orthogonal deprotection at a later stage in the synthesis. After Fmoc deprotection of the final residue, the N-terminus was acetylated using acetic anhydride (48 μL, 0.5 mmol) and diisopropylethylamine (43 μL, 0.25 mmol) in dimethylformamide for 2 hours and the Dde protection of the lysine side chain removed with 2% hydrazine in dimethylformamide for 15 mins. The free amine of the lysine side chain was extended by reaction with succinic anhydride (50 mg, 0.5 mmol) and diisopropylethylamine (43 μL, 0.25 mmol) in dimethylformamide for 2 hours and then hydrazine, coupled as a 10% solution in dimethylformamide using HBTU/HOBt (in excess) for 3 hours. Final cleavage of the peptide from the resin was performed with 92.5% trifluoroacetic acid/2.5% triisopropylsilane/2.5% water/2.5% ethanedithiol (40 mL/g resin) for 75 mins. The resin was removed by filtration and the filtrate was concentrated by sparging with nitrogen. The crude product was precipitated and washed with cold methyl tert-butyl ether (3×50 mL), before being re-dissolved in 50% (aq.) acetonitrile and lyophilised. The peptide was re-dissolved in ammonium bicarbonate (0.1 M, pH 8) to a concentration of 100 μM and oxidised using hydrogen peroxide (1.5 eq) for 45 mins. The reaction was monitored by LC-ESI-MS and with Ellman's reagent and finally quenched with 10% (aq) acetic acid (in excess). The mixture was lyophilised once more and then purified by semi-preparative RP-HPLC. Yield: 24 mg, 0.019 mmol, 37%. ESI-MS m/z: 1291.3 (calc. for M+H⁺ 1291.5). HPLC retention time: 3.44 mins.

### B. Synthesis of {5S-(Carboxymethylcarbamoyl)-5-[5-(4-formyl-3-hydroxy-phenoxy)pentanoyl amino]pentyl}trimethylammonium (14)

The compound was synthesised manually using Fmoc/tBu protection strategy on 2-chlorotrityl resin (0.19 g, 0.19 mmol), pre-loaded with glycine (substitution: 1.0 mmol/g). Fmoc-Lys(Me)₃—OH was double coupled using an HBTU/HOBt method with dimethylformamide as the solvent and 3 equivalents of amino acid and coupling reagents with respect to the loading of the resin. The Fmoc group was removed by a 15 min treatment with 20% piperidine in dimethylformamide. Coupling of 5-(4-formyl-3-hydroxyphenoxy) pentanoic acid (BAL) (16) was accomplished with a benzotriazole-1-yl-oxy-tris-(dimethylamino)phosphoniumhexafluoro phosphate (BOP) (BOP/HOBt) method utilising dimethylformamide as the solvent and 3 equivalents of (16) and coupling reagents with respect to the loading of the resin. A final 20% piperidine treatment was included to remove any ester formed at the 2-hydroxyl position of the BAL. Final cleavage of the linker from the resin was performed with several treatments of 5% trifluoroacetic acid in dichloromethane, each for 5 mins. The resin was removed by filtration and the pooled filtrate was concentrated by sparging with nitrogen. The crude product was precipitated and washed with cold methyl tert-butyl ether, before being re-dissolved in 30% (aq) acetonitrile and lyophilised. Finally, the compound was purified by semi-preparative RP-HPLC, the pure fractions pooled and lyophilised once more to yield an off white solid. Yield: 35 mg, 0.075 mmol, 39%. ESI-MS m/z: 466.2 (calc. for M+H⁺ 466.26). HPLC retention time: 3.75 mins.

### C. {5S—[(2,5-Dioxopyrrolidin-1-yloxycarbonylmethyl)carbamoyl]-5-[5-(4-formyl-3-hydroxyphenoxy)pentanoylamino]pentyl}trimethylammonium (15)

Compound (14) (35 mg, 0.075 mmol) was dissolved in dimethylformamide (2 mL) and added to a stirred solution of PS-carbodiimide (288 mg, 0.375 mmol) in dichloromethane (10 mL). The mixture was stirred for 20 mins before the addition of N-hydroxysuccinimide (9 mg, 0.075 mmol) dissolved in dimethylformamide (1 mL). The reaction was then stirred at room temperature and monitored by HPLC until completion (5 hours). The resin was removed by filtration, the solvent removed in vacuo and the compound used without further purification. Yield: 38 mg, 0.068 mmol, 90%. ESI-MS m/z: 563.3 (calc. for M+H+563.3). HPLC retention time: 4.16 mins.

### D. 5-(4-formyl-3-hydroxyphenoxy)pentanoic Acid (BAL) (16).

2,4-Dihydroxybenzaldehyde (10 g, 0.072 mol) and spray-dried potassium fluoride (8.4 g, 0.144 mol) were stirred vigorously at 60° C. for 20 mins in anhydrous acetonitrile (150 mL). Methyl-5-bromovalerate (42.3 g, 0.216 mol) was added in one portion and the mixture brought to a gentle reflux for 5 hours. The reaction was allowed to cool to room temperature and the solvent removed in vacuo. The residue was partitioned between water (100 mL) and ethyl acetate (50 mL). The aqueous was washed twice more with ethyl acetate (2×30 mL) and the combined organic back-washed with water, dried over anhydrous magnesium sulphate, filtered and evaporated to dryness. The resulting red oil was re-crystallised from ether (30 mL) and heptane (20 mL). The methyl ester obtained was dissolved in tetrahydrofuran (120 mL) and stirred vigorously at room temperature. To this solution was added lithium hydroxide (3.7 g, 0.088 mol) dissolved in water (60 mL) and the mixture stirred for 4 hours. The solvent was reduced in vacuo and the resultant oily residue diluted with water (30 mL), washed twice with methyl tert-butyl ether (2×50 mL), acidified to pH 2 with conc. hydrochloric acid and extracted with ethyl acetate (4×30 mL). The combined ethyl acetate was dried over anhydrous magnesium sulphate, filtered and evaporated to dryness to give a white solid product. Yield: 9.86 g, 0.041 mol, 57%. ¹H NMR (CDCl₃) δ: 11.26 (2H, br.s), 9.69 (1H, s), 7.41 (1H, d, J=8.6 Hz), 6.51 (1H, dd, J=8.6, 2.2 Hz), 6.40 (1H, d, J=2.2 Hz), 4.02 (2H, t, J=5.9 Hz), 2.44 (2H, t, J=7.0 Hz), 1.84 (4H, m). mp: 88-91° C. ESI-MS m/z: 239.1 (calc. for M+H⁺ 239.08). HPLC retention time: 5.34 mins.

### E. 5-(4-formyl-3-hydroxyphenoxy)pentanoic acid 2,5-dioxopyrrolin-1-yl Ester (BAL-OSu) (17)

PS-carbodiimide resin (4.2 g, 5.5 mmol) was suspended in dichloromethane (45 mL) stirred for 5 mins to swell the resin. Compound (16) (1.0 g, 4.2 mmol) was added, dissolved in dichloromethane (10 mL) and the resin mixture stirred for a further 20 mins before the addition of N-hydroxysuccinimide (0.46 g, 4.0 mmol) dissolved in dimethylformamide (4 mL). The reaction was then stirred at room temperature and monitored by HPLC until completion (18 hours). The resin was removed by filtration, the solvent removed in vacuo and the final product re-crystallised from isopropanol.

Yield: 1.3 g, 3.8 mmol, 92%. ESI-MS m/z: 336.1 (calc. for M+H⁺ 336.1). HPLC retention time: 6.18 mins.

### F. [2S-[5-(4-formyl-3-hydroxyphenoxy)pentanoylamino]-6-(2,2,2-trifluoroacetyl amino)hexanoylamino]acetic Acid (Tfa) (18)

The compound was synthesised manually by solid phase synthetic methods, using Fmoc/tBu protection strategy on 2-chlorotrityl resin (0.3 g, 0.3 mmol), pre-loaded with glycine (substitution: 1.0 mmol/g). Coupling of Fmoc-Lys(Tfa)-OH was accomplished with a 2-(1H-benzotriazole-1-yl)-1,1,3,3-tetramethyluroniumhexafluorophosphate/N-hydroxybenzotriazole (HBTU/HOBt) method utilising dimethylformamide as the solvent, using 3 equivalents of amino acid and coupling reagents with respect to the loading of the resin. The Fmoc group was removed by a 15 min treatment with 20% piperidine in dimethylformamide. Coupling of 5-(4-formyl-3-hydroxyphenoxy) pentanoic acid (BAL) (16) was achieved as above using BOP activation. A final 20% piperidine treatment was included to remove any ester formed at the 2-hydroxyl position of the BAL. Final cleavage of the linker from the resin was performed with several treatments of 5% trifluoroacetic acid in dichloromethane, each for 5 mins. The resin was removed by filtration and the pooled filtrate was concentrated by sparging with nitrogen. The crude product was precipitated and washed with cold methyl tert-butyl ether, before being re-dissolved in 50% (aq) acetonitrile and lyophilised. Finally, the compound was purified by semi-preparative RP-HPLC, the pure fractions pooled and lyophilised once more to yield a white solid. Yield: 49 mg, 0.095 mmol, 32%. ESI-MS m/z: 520.2 (calc. for M+H⁺ 520.1). HPLC retention time: 5.12 mins.

### G. [2S-[5-(4-formyl-3-hydroxyphenoxy)pentanoylamino]-6-(2,2,2-trifluoroacetyl amino)hexanoylamino]acetic Acid 2,5-dioxopyrrolin-1-yl Ester (Tfa-OSu) (19)

Compound (18) (49 mg, 0.095 mmol) was dissolved in dimethylformamide (2 mL) and added to a stirred solution of PS-carbodiimide (375 mg, 0.475 mmol) in dichloromethane (10 mL). The mixture was stirred for 20 mins before the addition of N-hydroxysuccinimide (11 mg, 0.095 mmol) dissolved in dimethylformamide (1 mL). The reaction was then stirred at room temperature and monitored by HPLC until completion (5 hours). The resin was removed by filtration, the solvent removed in vacuo and the compound used without further purification. Yield: 55 mg, 0.089 mmol, 93%. ESI-MS m/z: 617.2 (calc. for M+H⁺ 617.1). HPLC retention time: 5.64 mins.

### EXAMPLE 2

### Solubility Studies with BSA-linker Constructs

Fluram Assay.

Test sample or standard (10 μL) was added to an assay plate well containing di-basic sodium hydrogen phosphate buffer (85 μL). Fluram was dissolved in acetonitrile (1 mg/mL) and 5 μL of this solution was added to each well, mixed and allowed to react for 5 mins before a fluorescence reading was obtained.

Stoichiometric Evaluation of BSA Acylation.

BSA was dissolved in 0.1 M sodium acetate (pH 7.25) to produce a 10 mg/mL solution, of which 10 μL was transferred (in triplicate) into wells containing 160 μL di-basic sodium hydrogen phosphate buffer (0.1 M, pH 8.2). 85 μL of the samples was transferred across the plate with double dilution into di-basic sodium hydrogen phosphate buffer. Fluram was dissolved in acetonitrile (20 μg/mL) and 5 μL (0.1 μg, 359 pmol) of this solution was added to each well, mixed and allowed to react for 5 mins before a fluorescence reading was obtained.

Preparation of BSA-Linker Constructs (20,21,22).

BSA (2 mg, 29 mmol) was dissolved in 0.1 M sodium acetate (1 mL, pH 7.25) and added to BAL-OSu (17) (2.5 mg, 7.46 μmol), Tfa-OSu (19) (4.6 mg, 7.46 μmol) or TML-OSu (15) (4.2 mg, 7.46 μmol) each dissolved in dimethyl sulfoxide (0.5 mL). The reactions were stirred at room temperature and the disappearance of free amine monitored with Fluram. Once complete (approx. 2-3 hours), the reaction mixtures were dialysed (3×2 L) against 10 mM ammonium bicarbonate (pH 8) and the products analysed by gel electrophoresis.

Bradford Assay.

A standard BSA solution (0.5 mg/mL) was prepared and a range of volumes (0-15 μL) added to wells containing water to give a total volume of 100 μL. In a similar manner, 5 μL of the test sample was added to wells (in triplicate) containing water (95 μL). Bradford reagent (100 μL) was then added to both standard and test wells and the solutions mixed with a multi-channel pipette. The plate was then left at room temperature for 5 mins before UV measurements were taken. Protein concentrations were determined by comparison with the standard curve generated for BSA.

Solubility Measurements of BSA-Linker Constructs (20,21,22) at pH 8.

1 mL of the BSA-linker constructs (20,21,22) in ammonium bicarbonate buffer was concentrated to approximately a fifth of its original volume by centrifugal filtration and the protein concentration assessed by a Bradford assay. The concentration of the BSA-linker constructs (20) and (22) was adjusted to 5 mg/mL while the preparation derived from (21) was adjusted to 4 mg/mL. 40 μL of each solution was transferred into wells (in triplicate) of a 384-well microtitre plate and 20 μL of each sample was transferred across the plate with double dilution into ammonium bicarbonate buffer. The samples were allowed to come to equilibrium over 30 mins before turbidity measurements were taken.

Solubility Measurements of BSA-Linker Constructs (20,21,22) at pH 4.5.

1 mL volumes of the BSA-linker constructs (20,21,22) in ammonium bicarbonate buffer were concentrated to approximately a fifth of their original volume by centrifugal filtration. These filters were then employed in solvent exchange process to replace the original ammonium bicarbonate buffer with a sodium formate buffer (0.1 M, pH 4.5). This was achieved through cycles of dilution and concentration with the new buffer (approx. 5-6 cycles) until the theoretical ammonium bicarbonate content was below 1%. The protein content of the concentrated preparations (now in formate buffer) was then assessed by a Bradford assay. 40 μL of each solution was transferred into wells (in triplicate) of a 384-well microtitre plate and 20 μL of each sample was transferred across the plate with double dilution into sodium formate buffer (0.1 M, pH 4.5). The samples were allowed to come to equilibrium over 30 mins before turbidity measurements were taken.

Results and Discussion

Solubility of BSA-Linker Constructs (20-22). In order to assess the approximate number of free amines in bovine serum albumin (BSA) carrier protein, that were available for conjugation, a stoichiometric evaluation of the reaction between an amine specific fluorescent reagent (Fluram) and BSA was performed. By keeping one reactant constant and gradually increasing the other, a plateau was reached indicating a point of equivalence (FIG. 1). Since the number of free amines in BSA is known an estimate of the number taking part in the reaction was made (approx. 21-25).

Three BSA-linker constructs containing TML85 (20), Tfa85 (21) and BAL85 (22) were initially prepared, via coupling with activated linkers (15, 17, 19), to approximately 85%-90% loading of accessible surface amines (estimated by Fluram monitoring). Characterisation of the BSA modified constructs by gel electrophoresis confirmed the expected increase in molecular weight compared with the native BSA (FIG. 2).

The BSA-TML85 (20) construct proved a highly modified protein that retained good aqueous solubility over a wide pH range, whereas BSA constructs derived from the BAL and Tfa linkers were less soluble. At pH 8, BSA-TML85 (20) and BSA-BAL85 (22) showed reasonably good solubility at around 2-3 mg/mL, while BSA-Tfa85 (21) precipitated around 0.5 mg/mL (FIG. 3).

At more acidic conditions (pH 4.5) BSA-BAL85 (22) and BSA-Tfa85 (21) exhibited low solubility and precipitated at concentrations above 250 μg/mL, whereas BSA-TML85 (20) possessed solubility well above 3.5 mg/nL (FIG. 4). This is a particularly important finding since, as detailed earlier, the conjugation reaction between construct and epitope is chemoselective at acidic pH, (ideally performed at pH 4-4.5). Thus poor solubility of BSA-linker constructs at acidic pH is extremely detrimental in the formation of highly loaded BSA conjugates.

### EXAMPLE 3

### Solubility Studies with BSA-Linker-Epitope Constructs Preparation of BSA-Linker Construct (23)

BSA (2 mg, 29 nmol) was dissolved in 0.1 M sodium acetate (1 mL, pH 7.25) and added to BAL-OSu (17) (0.25 mg, 0.746 μmol) dissolved in dimethyl sulfoxide (0.5 mL). The reaction was stirred at room temperature and the disappearance of free amine monitored with Fluram until approx. 55% acylation had been achieved (approx. 2 hours). The reaction mixture was dialysed (3×2 L) against 10 mM ammonium bicarbonate (pH 8) and the products analysed by gel electrophoresis.

Hydrazone Conjugation of Epitope (13) to BSA-Linker Constructs (20,23) Providing Conjugates (24,27)

2 mL of BSA-linker constructs (20) and (23) in ammonium bicarbonate buffer were dialysed (3×4 L) against sodium formate buffer (0.1 M, pH 4) producing a final concentration of approx. 1 mg/mL. Oxytocin analogue (13) (2.6 mg, 2.1 μmol) was dissolved in dimethyl sulfoxide (1.6 mL) and added to the BSA-linker construct solutions (2 mL), the final content of dimethyl sulfoxide being approximately 45%. The solutions were stirred at room temperature for 18 hours and dialysed (3×2 L) against 10 mM PBS (pH 7.4). The conjugates (24) and (27) were characterised by gel electrophoresis.

Solubility Measurements of BSA Conjugates (24,27).

1 mL volumes of the BSA conjugates (24,27) in 10 mM phosphate buffer, at the chosen pH, were concentrated to approximately a fifth of their original volume by centrifugal filtration and the protein content of the concentrated preparations was then measured by a Bradford assay. 40 μL of each solution was transferred into wells (in triplicate) of a 384-well microtitre plate and 20 μL of each sample was transferred across the plate with double dilution into the phosphate buffer. The samples were allowed to come to equilibrium over 30 mins before turbidity measurements were taken.

Results and Discussion

Preparation and Solubility of BSA-Linker-Epitope Conjugates (24-27).

The neurohypophysial hormone oxytocin, is a disulfide constrained nonapeptide (cyclo-[CYIQNC]PLG), and was chosen as a model epitope with which to carry out conjugation and immunisation studies. Typically, the conjugation reactions between BSA-linker constructs (20-22) and epitope (13) were performed in an aqueous buffer/dimethyl sulfoxide medium at pH4-4.5. Loading reactions were complete after approximately 18 hours, using 2-3 equivalents of the oxytocin analogue hydrazide (13) with respect to the number of moles of aldehyde accessible for conjugation. Initially, only conjugates BSA-TML85-epitope13 (24) and BSA-BAL85-epitope13 (26) were adequately prepared since the poor solubility of the BSA-Tfa85 (21) construct hampered any synthetic efforts to produce the conjugate BSA-Tfa85-epitope13 (25). Upon dialysis into phosphate buffer (pH 7.4), however, the conjugate obtained from BSA-BAL85-epitope13 (26), precipitated and aggregated becoming very poorly soluble. In contrast, the conjugate BSA-TML85-epitope13 (24) remained relatively soluble with only a slight precipitate seen in the solution. In order to proceed with immunisation studies, a soluble conjugate based around BAL linker (17) was required and such a conjugate, BSA-BAL55-epitope13 (27), was obtained with a reduced surface loading of approximately 55%, through BSA-BAL55 (23). Solubility studies showed that the BSA-TML85-epitope13 (24) and BSA-BAL55-epitope (27) conjugates had good solubility at pH 6 and pH 7.4 of around 0.5-1 mg/mL (FIGS. 5 and 6).

Gel electrophoresis confirmed the increase in molecular weight compared with the native BSA (FIG. 7).

### EXAMPLE 4

### Chemical Analysis of BSA-Linker-Epitope Constructs

Hydrolysis of BSA-TML-Oxytocin Conjugate (24).

Equal volumes of conjugate and 1N hydrochloric acid were mixed and assayed by LC-MS every 15 mins.

Results and Discussion

Hydrolysis of BSA-TML85-epitope13 conjugate (24). Reversibility of the linkage between carrier protein and epitope is fundamentally crucial to quality control of the conjugate production process. Unless the chemical integrity of the loaded epitope can be confirmed post-conjugation, the validity of any results obtained with the conjugate must be treated with caution.

Acid hydrolysis of the hydrazone bond within BSA-TML85-epitope13 conjugate (24) regenerated BSA-TML85 (20) and epitope (13). The reaction progressed smoothly over 1 hr, with clear identification of epitope (13) by LC-ESI-MS (FIG. 8). Analysis for a free thiol within this hydrolysis product with Ellman's reagent proved negative, confirming the integrity of the disulfide bond within epitope (13).

### EXAMPLE 5

### Immunisation Studies

Mice were immunised with 50 μg of BSA alone or with oxytocin conjugated to BSA using either BAL linker or TML linker, in complete Freund's adjuvant. The mice were then boosted on days 14 and 28 with 50 μg of the appropriate compound in incomplete Freund's adjuvant before final bleeds were harvested on day 42. The ELISA analysis was carried out in Nunc-immuno plates and coated with free oxytocin peptide. Casein was used as a blocking solution to prevent non-specific interaction of antibody with the microtitre plate. The plates were developed using an alkaline phosphatase linked anti-mouse IgG secondary antibody with disodium p-nitrophenyl phosphate and the absorbance of each well was read at 405 nm.

The results are shown in FIGS. 9-11. FIG. 9 shows that both constructs are recognised by antibodies raised to BSA alone, which is to be expected since BSA is the carrier protein present within both constructs, and thus provides a positive control.

Comparison of the antibody titres raised against the BSA-BAL55-oxytocin construct and the BSA-TML85-oxytocin construct reveals distinct differences in the nature of the antibodies produced. Results from mice immunised with the BSA-BAL55-oxytocin construct (FIG. 10) show a greater proportion of BSA (non-specific) antibodies produced than those antibodies specific for the whole construct itself. In contrast, mice immunised with the BSA-TML85-oxytocin construct (FIG. 11) show the converse; the proportion of specific antibodies raised to the whole construct is greater than the non-specific BSA titres.

These results show that the TML85 construct has greater epitope surface coverage and greater aqueous solubility than the BAL55 construct. This is supported by the results of Examples 1 to 4 described above.

### EXAMPLE 6

### Preparation and Spectrophotometric Characterisation of TML Linker (14) Reaction with Proteins

(a) Chemical Coupling of Various Proteins with TML Linker (14) Providing Compounds of General Formula (XIV)

Unless otherwise stated all the proteins were purchased from the Sigma Chemical Company, Poole, Dorset. Aprotonin (APRO; cat # A4520), Hen egg-white lysozyme (HEWL; cat # L6876), carbonic anhydrase (cat # C2273), ovalbumin (OVA; cat # A7642), bovine serum albumin (BSA; cat # A7638), apo-transferrin (cat # T4382); holo-transferrin (cat #T4132); phosphorylase B (phosB; cat # P4649), β-galactosidase (β-gal; cat # G8511) and myosin (MYO; cat # M3889) were made up to 1 mg/ml in 50 mM potassium phosphate, pH 9.3. The amount of reactive amine for each protein sample was determined using the Fluram assay (as described above), employing Fmoc-Lys-OH (Novabiochem) as the calibration control. These data were used as the basis for determining the reactive amine stoichiometry for each protein.

TML linker (14) was coupled onto each of the proteins by mixing various amounts of 10 mM TML-NHS (15) in DMSO with 200 μl of a 1 mg/ml solution of each of the proteins dissolved in 50 mM potassium phosphate; pH 9.3. To aprotinin was added 438 μl 10 mM TML-NHS (15); to HEWL was added 135 μl 10 mM TML-NHS (15); to carbonic anhydrase was added 38 μl 10 mM TML-NHS (15); to ovalbumin was added 42 μl 10 mM TML-NHS (15); to BSA was added 65 μl mM TML-NHS (15); to Apo-transferrin was added 80 μl 10 mM TML-NHS (15); to holo-transferrin was added 80 μl 10 mM TML-NHS (15); to phosphorylase B was added 15 μl 10 mM TML-NHS (15); to β-gal was added 71 μl 10 mM TML-NHS (15) and to myosin was added 100 μl 10 mM TML-NHS (15). The samples were incubated at room temperature for 60 min. followed by overnight incubation in the fridge (˜8° C.).

The TML-NHS (15)-protein reaction mixtures were subdivided into three equivalent volumes for further processing. One portion of each of the TML-NHS (15)-protein reaction mixtures was dialysed, in benzoylated dialysis tubing (SpectraPor 1.2 kDa cut-off membrane; Sigma cat # D2272), as one batch against three changes of 1800 ml 10 mM sodium acetate; pH 7.25. The first two procedures were carried out for 60 min. each followed by a further overnight dialysis cycle. The samples were subsequently dialysed as a batch against 1800 ml of 10 mM sodium formate; pH 4.0 for 60 min. The samples were recovered and stored in the fridge until required.

(b) Chemical Coupling of Hepatitis B Virus Core Delta Antigen (HBV Core Delta Ag) with TML Linker (14) and Reaction with Biotin Hydrazide

Recombinant HBV core delta Ag, strain ayw, was purchased from Advanced ImmunoChemical Inc., Long Beach, Calif., USA. To 75 μl of a 1 mg/ml solution of HBVcoreΔAg was added 7.5 μl 0.5 M sodium phosphate; pH 9.3. The sample was mixed and from this pool, an aliquot (55 μl) was removed and 5.5 μl 10 mM TML-NHS (15) added to it, the sample mixed by pipetting and incubated at room temperature for 2 h. After this incubation period, a further 27.5 μl was removed and 2 μl 2.65 M formic acid added to the sample. The sample was mixed and 1.37 μl of 10 mM biotin-hydrazide was added to the sample. This sample was incubated at room temperature for 60 min. and then overnight at ˜8° C. The various protein samples were analysed as described below.

(c) Gel-Analysis of TML-Labelled Proteins.

The dialysed TML-NHS (15)-protein samples were recovered and analysed by SDS-PAGE employing the NuPAGE system (Invitrogen) using a 4-12% bis-tris NuPAGE gel with MES running buffer. Proteins were visualised with SilverExpress stain kit. The protocols were carried out according to the manufacturers instructions (FIG. 14).

(d) Production of BSA- and Aprotinin-TML Conjugated Proteins for UV Measurements

BSA (20 mg) was dissolved in 2 ml 50 mM potassium phosphate; pH 9.3 and the sample dialysed (10 kDa molecular weight cut-off, Slidealyser; Perbio) against 5 L 50 mM potassium phosphate; pH 9.3 for 60 min. at room temperature. The protein sample was recovered and while stirring 200 μl 10 mM TML-NHS (15) in 100% DMSO was added. The sample was stirred at room temperature for 20 min. after which a further 200 μl TML-NHS (15) added and the sample stirred. A further 234 μl TML-NHS (15) was added after 20 min. and the sample stirred for a further 30 min. The TML-NHS (15) treated sample was recovered and dialysed as before against 5 L 100 mM sodium formate; pH 3.5 for 60 min. after which it was dialysis buffer was changed for fresh 100 mM sodium formate; pH 3.5 and the sample dialysed overnight at room temperature. The protein-TML sample was recovered (˜3 ml) and centrifuged at 13,000 r.p.m. for 10 min. and the supernatant collected. This was treated as the BSA-TML sample.

Aprotinin (2 mg) was dissolved in 2 ml 50 mM potassium phosphate; pH 9.0 and the sample dialysed (1 kDa molecular weight cut-off; SpectraPor; Sigma) overnight against 5 L 50 mM potassium phosphate; pH 9.0 at room temperature. The protein sample was recovered (˜1.8 ml) and while stirring 100 μl 10 mM TML-NHS (15) in 100% DMSO was added. The sample was stirred at room temperature for 120 min. after which the sample was dialysed as before against two changes of 5 L 100 mM sodium formate; pH 3.5 for 60 min. and 120 min. respectively. The protein-TML sample was recovered (˜3 ml) and centrifuged at 13,000 r.p.m. for 5 min. and the supernatant collected. This was treated as the Aprotinin-TML sample.

(e) Characterisation of Absorbance Spectra of TML Linker (14) Conjugated Proteins

Aliquots (10 μl) of BSA-TML and Aprotinin-TML were diluted with 190 μl 50 mM potassium phosphate buffer ranging from pH 6.0 to pH 11.0. The buffers were made up to the required pH by mixing appropriate portions of 50 mM di-potassium hydrogen phosphate and 50 mM potassium di-hydrogen phosphate. Where require, sodium hydroxide was used to adjust the pH. The absorption spectra were collected in UVStar 96-well microtiter plates (Greiner) using a Spectramax384 instrument (Molecular Devices, Crawley, U.K.) (see FIG. 12).

(f) Characterisation of Fluorescence Spectra of TML Linker (14) Conjugated Proteins

Aliquots (10 μl) of post-dialysis buffer (100 mM sodium formate; pH 3.5), BSA-TML and Aprotinin-TML were diluted with 190 μl 500 mM potassium phosphate buffer ranging from pH 12.0. The fluorescence emission spectra were collected in Microfluor W 96-well microtiter plates (Thermo Dynex) using a Gemini instrument (Molecular Devices, Crawley, U.K.) (see FIG. 13).

### EXAMPLE 7

### Chemoselective Addition of Ligands to Linker-Proteins (XIV) to Provide Protein Constructs (XV)

(a) Reaction of Biotin Hydrazide with Protein-TML Compounds of General Formula (XIV) Providing Compounds of General Formula (XV).

To the 10 mM sodium formate; pH 4.0 dialysed portions of each of the protein-TML reaction mixtures (ex Example 6(a)) was added varying amounts of 10 mM biotin-hydrazide (Aldrich cat # 14403; in DMSO). To 133 μl aprotinin-TML reaction mixture was added 26.6 μl 10 mM biotin-hydrazide; to 112 μl HEWL-TML reaction mixture was added 22 μl 10 mM biotin-hydrazide; to 79 μl carbonic anhydrase-TML reaction was added 15 μl 10 mM biotin-hydrazide; to 80 μl ovalbumin-TML reaction mixture was added 15 μl 10 mM biotin-hydrazide; to 88 μl BSA-TML reaction mixture was added 15 μl 10 mM biotin-hydrazide; to 71 μl phosphorylase B-TML reaction mixture was added 15 μl 10 mM biotin-hydrazide; to 90 μl β-gal-TML reaction mixture was added 15 μl 10 mM biotin-hydrazide and to 66 μl myosin-TML reaction mixture was added 15 μl 10 mM biotin-hydrazide. The samples were incubated at room temperature for 60 min. and then stored in the fridge (˜8° C.) for seven days.

For the transferrin experiments, to 140 μl apo-transferrin-TML and 140 μl holo-transferrin-TML, both in 50 mM potassium phosphate; pH 9.0 buffer, was added 140 μl 0.2 M sodium formate; pH 4.0. The samples were mixed and 100 μl 10 mM biotin-hydrazide added to both samples. The reactions were incubated at room temperature for 2 h and then overnight at ˜8° C.

After incubation, all the reaction mixtures were recovered and each dialysed, in benzoylated dialysis tubing (SpectraPor 1.2 kDa cut-off membrane; Sigma cat # D2272), together against 4000 ml 20 mM sodium acetate; pH 7.4 for 120 min. at room temperature. The samples were recovered and stored in the fridge until further analysis.

(b) Gel Shift and Western Blot Analysis of Protein-TML-Biotin Constructs (XV)

The dialysed protein-TML-biotin samples (ex Example 7(a)) were recovered and analysed by SDS-PAGE employing the NuPAGE system (Invitrogen) using a 4-12% bis-tris NuPAGE gel with MES running buffer. Proteins were transferred onto a PVDF membrane using the Novex blot transfer system (Invitrogen). The protocols were carried out according to the manufacturers instructions.

The PVDF membrane was blocked by gentle agitation of the membrane in 50 ml phosphate buffered saline containing 1% Tween 20 (PBST; Sigma cat # P3563) containing 1% (w/v) BSA for 15 min. The recovered membrane was washed three times, by gentle agitation, in 100 ml PBST for 5 min. per cycle. The recovered membrane was then incubated in 50 ml PBST containing 1:5000 dilution of ExtrAvidin®-Peroxidase (Sigma E2886) for 30 min. The membrane was recovered, washed three times in PBST and allowed to partially drip-dry. Regions of peroxidase activity were visualised by addition of a 3,3′,5,5′-etramethylbenzidine (TMB) liquid substrate (Sigma cat # T0565) onto the static membrane. After appropriate exposure, the membrane was recovered, washed in water and air dried prior to analysis and storage (see FIGS. 14 and 15).

(c) Texas Red® Labelling of TML Activated Proteins.

The dialysed protein-TML samples (ex Example 6(a))) were recovered and to 10 μl of each sample was added 2 μl DMSO and 1 μl 10 mM Texas Red®-hydrazide (Molecular Probes). The samples were mixed and incubated at room temperature for 3 h. Subsequently, 2.5 μl of sample loading buffer (Novex) and 1.25 μl sample-reducing buffer (Novex) were added to each sample. The samples were analysed by SDS-PAGE employing the NuPAGE system (Invitrogen) using a 4-12% bis-tris NuPAGE gel with MES running buffer. Protocols were carried out according to manufacturers instructions. Protein bands were visualised by eye.

(d) ELISA Analysis of Biotin Derivatised Compounds of General Formula (XV)

Enzyme-linked immunosorbent assay (ELISA) of BSA-TML and aprotinin-TML samples reacted with biotin-hydrazide was carried out as follows. Aliquots (1 μl) of each quenched sample were diluted into 500 μl 10 mM phosphate buffer saline (PBS; Sigma) and 100 μl of this dispensed into a 96-well Immulon 2HB microtitre plate (Thermo Life Sciences, Basingstoke, U.K.). The plate was covered and incubated at 37° C. for 60 min. after which it was washed three cycles of 200 μl of PBS containing Tween 20 (PBST; Sigma) per cycle. The plate was shaken dry by hand and 100 μl 1 in 5000 dilution of ExtrAvidin-HRP conjugate (Sigma) added to each well. The plate was covered and incubated at 37° C. for 30 min. The plate was recovered and washed as before, dried by shaking and 100 μl OPD reagent (Sigma) added to each well. The colour was allowed to develop by eye and the reaction stopped by addition of 100 μl 0.1 M sulphuric acid. The plates were read at 492 nm (Spectramax 384) to quantify the colour reaction (see FIG. 18).

### EXAMPLE 8

### Spectrophotometric Assessment of Chemoselective Addition of Ligands to Linker-Proteins (XIV) to Provide Protein Constructs (XV)

(a) Spectrophotometric Characterisation of the Reaction of Biotin-Hydrazide with TML-Conjugated Proteins

To an aliquot (100 μl) of BSA-TML and aprotinin-TML was added an equivalent volume of 10 mM biotin-hydrazide (dissolved in 100 mM sodium formate; pH 3.5) and the reaction mixed. The sample was divided, with one portion being used for collection of the absorption spectra as a function of time (see FIGS. 16 and 17) as the second portion was concomitantly sampled by withdrawing and aliquot (10 μl) at the end of each data collection and quenched into 4×LDS buffer (NuPAGE loading buffer; Invitrogen). The quenched samples were frozen (minus 20° C.) until required. The quenched protein samples were analysed by SDS-PAGE (NuPAGE system) and Western blot as described above. To facilitate direct comparisons between proteins samples upon gel electrophoresis and staining, the BSA-TML samples and aprotinin-TML samples were mixed appropriately prior to loading onto the gels.

### EXAMPLE 9

### Characterisation of the Cleavage Reaction of Protein-TML-Ligand Constructs

To an aliquot (200 μl) of BSA-TML-biotin was added 200 μl 200 mM formic acid; pH 2.1 and the reaction mixed. The sample was further acidified by the addition of 20 μl 12.1 M HCl, the sample mixed and divided, with one portion being used for collection of the absorption spectra as a function of time (see FIGS. 20) as the second portion was concomitantly sampled by withdrawing and aliquot (20 μl) at the end of each data collection and quenched into 4×LDS buffer (NuPAGE loading buffer; Invitrogen). The quenched samples were frozen (minus 20° C.) until required. The quenched protein samples were analysed by SDS-PAGE (NuPAGE system) and Western blot as described above (FIGS. 21 and 22).

### EXAMPLE (10)

### Derivatisation of Solid Phase Surfaces Using TML Linker (15) and Reaction with Biotin Hydrazide

(i) Glass slides (Fisher Scientific; Menzel Superfrost 76×26 mm, ISO-Norm 8037) were cleaned and amine functionalised by derivatisation with amino-propylsilane (Acros, cat # 15181000) as described previously (MacBeath, et al., (1999), J. Am. Chem. Soc., 121, 7967-7968). The amine-functionalised slides were stored at room temperature until required.

(a) Slide Derivitisation with TML Linker (14)

Amine-functionalised slides were elaborated with TML linker by spotting 10 mM TML-NHS (15) in 100% DMSO onto the glass surface using a blunt-end syringe needle (Rheodyne). The spotted slides were covered and incubated at room temperature for 60 min. The slides were subsequently washed using the following cycle: ˜10 mL DMSO, ˜10 mL water, ˜10 mL methanol, ˜10 mL DMSO and ˜10 mL 10 mM potassium phosphate; pH 7.4. The TML linker elaborated slides were stored at room temperature until required.

(b) Derivitisation of Glass-TML Linker with Biotin Hydrazide

A TML linker elaborated slide was covered with 100 μM biotin hydrazide dissolved in 0.2 M sodium formate; pH 4 containing 50% (v/v) DMSO. The slide was covered and incubated for 30 min. at room temperature. The slide was recovered, washed thoroughly with water (˜20 mL) followed by methanol (˜20 mL) and then sonicated in 40 mL of methanol twice for 2 min. per cycle. The slide was recovered, a drop of 1 M HCl was placed onto the middle of the slide for 15 min. at room temperature. The slide washed with water, air-dried and inserted into a 50 mL Falcon tube containing 50 mL PBST containing 1:5000 dilution of ExtrAvidin-HRP conjugate (Sigma). The tube was gently rolled on a roller-bed for 15 min. at room temperature. The slide was recovered and washed three times in 40 mL PBST for 5 min. per cycle. The slide was recovered and washed in water and then air-dried. Approximately 1 mL of TMB liquid peroxidase substrate (Sigma) was dropped onto the slide and the colour was allowed to develop by eye (see FIG. 23).

(ii) Derivatisation of 96-well plates

(a) Derivitisation of Reacti-Bind Microtitre Plates with TML Linker (14).

Wells of a maleic anhydride activated polystyrene 96-well microtitre plate (Reacti-Bind Plates, Perbio Sciences UK Ltd., Tattenhall, U.K.) were amine functionalised by coupling 1,4-diaminobutane as a 1 mg/mL solution in 5% sodium carbonate, containing 60% DMSO (v/v), for 2 hours at 37° C. Following copious washing with DMSO, water and 5% sodium carbonate, TML-NHS linker (15) was coupled to some of the now amine functionalised wells using a 5 mM solution of the activated linker in 0.1 M sodium acetate; pH 7.25 containing 50% (v/v) DMSO for 2 hours at room temperature. A proportion of the amine functionalised wells were treated with a blank solution of only 0.1 M sodium acetate; pH 7.25 containing 50% (v/v) DMSO to provide controls. The wells were then once again washed with copious amounts of DMSO, 0.1 M sodium acetate; pH 7.25 and water and stored at room temperature until required.

(b) Derivitisation of TML Linker Functionalised Reacti-Bind Microtitre Plates with Biotin Hydrazide.

Biotin hydrazide was coupled to the TML linker functionalised Reacti-Bind plates as a 1 mM solution in 0.2 M sodium formate; pH 3.5 containing 50% DMSO (v/v). The coupling solution was also added to control wells not previously treated with TML linker. After 2 hours at room temperature, each well was washed with DMSO (1×200 μL), water (1×200 μL) and phosphate buffered saline containing tween 20 (PBST) (3×200 μL). 100 μL PBST was then added to each well and the plate incubated at 37° C. for 30 minutes in order to block any unreacted sites in the wells. After further washing with PBST (3×200 μL/well) 100 μL of ExtrAvidin-HRP conjugate (Sigma)(1:10000 dilution in PBST) was added and the wells incubated at 37° C. for a further 30 minutes. The wells were again washed with PBST (3×200 μL) before addition of 100 μL of o-phenylenediamine (OPD) peroxidase substrate (Sigma). The colorimetric reaction was left to develop by eye and finally quenched by the addition of 0.1 M sulphuric acid (100 μL) (FIG. 23).

### EXAMPLE 11

### Capture of ExtrAvidin-HRP from Solution Using TML-Resin

EAH Sepharose CL-4B (2 ml; 7-12 μmol/ml amine; APBiotech, Amersham, U.K.) was washed, in a scintered plastic column, with copious amounts of water followed by water:methanol (50:50), methanol and finally dimethylformamide (DMF). To the washed resin was added 900 μl 10 mM TML-NHS (15) in DMF. The reaction was incubated at room temperature for 60 min. and then the resin washed with DMF. To an aliquot (0.5 ml) of the TML treated resin was added biotin-hydrazide (4.5 μmol) in 2 ml DMF. The reaction was incubated at room temperature for 60 min. and then the resin washed with PBST (˜40 ml).

The PBST washed resin was incubated with a 50 ml solution of a 1 in 5000 dilution of ExtrAvidin-HRP (Sigma) for 30 min. at room temperature. The resin was recovered and washed, as before, with PBST. The resin was allowed to run dry under gravity and a small sample withdrawn using a glass pipette and dispensed into a Glasstic Slide 10 (Hycor Biomedical, Garden Grove, Calif., U.S.A.) microscope slide. An aliquot of TMB HRP substrate (Sigma) was introduced into the slide chamber and the colour reaction allowed to develop. During colour development images were captured using a microscope (QX3 CCD camera microscope; Intel) (FIG. 24).

1. A positive charge-balanced linker according to general formulae (Ia to Ie): 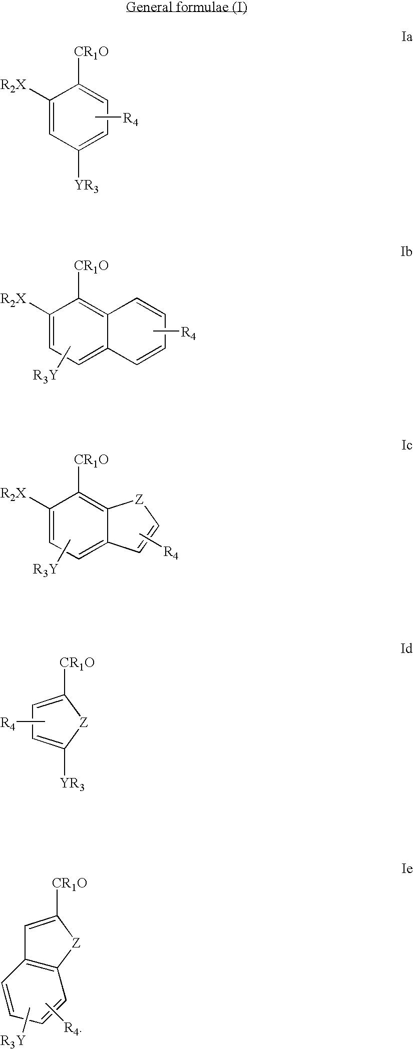
wherein:
X═O or S;
Y is O, S or CH₂, CHR, CRR, where R is C₁₋₇ alkyl;
Z is O or S;
R₁ is H or C₁₋₇ alkyl;
R₂ is H or C₁₋₇ alkyl;
R₄ is H or C₁₋₇ alkyl at any vacant position on the aromatic ring;
R₃ is C₁₋₇ alkyl-L₁-R₅-L₂-R₆—COOH, C₃₋₁₀ cycloalkyl-L₁-R₅-L₂-R₆—COOH or Ar—C₀₋₇ alkyl-L₁-R₅-L₂-R₆—COOH;
each of L₁ and L₂ is absent or is a linker selected from the group consisting of an amide CONH; an ether —O—; a thioether —S; and a sulphone —SO₂—;
R₅ is C₁₋₇ alkyl, C₃₋₁₀ cycloalkyl or Ar—C₀₋₇ alkyl each of which is substituted with either NR₈R₉, where the nitrogen atom is capable of being protonated in solution to give N⁺HR₈R₉; or a quaternary nitrogen atom N⁺R₈R₉R₁₀, such that R₅ contains a positive charge;
each of R₈, R₉ and R₁₀ is independently C₁₋₇ alkyl, C₃₋₁₀ cycloalkyl or Ar—C₀₋₇ alkyl, or any two or more of R₈, R₉ and R₁₀ together form an alicyclic or arylalicyclic ring system;
R₆ is C₁₋₇ alkyl, C₃₋₁₀ cycloalkyl or Ar—C₀₋₇ alkyl;
or a salt, hydrate, solvate, complex or prodrug thereof.

2. A compound as claimed in claim 1 wherein, independently or in any combination:
X is oxygen;
Y is oxygen;
R₁ is hydrogen, methyl or ethyl;
R₂ is hydrogen or C₁₋₄ alkyl;
L₁ is an amide (CONH); and
L₂ is an amide (CONH).

3. A compound as claimed in claim 1, wherein R₁ is hydrogen.

4. A compound as claimed in claim 1, wherein R₂ is hydrogen or methyl.

5. A compound as claimed in claim 1 wherein R₃ comprises 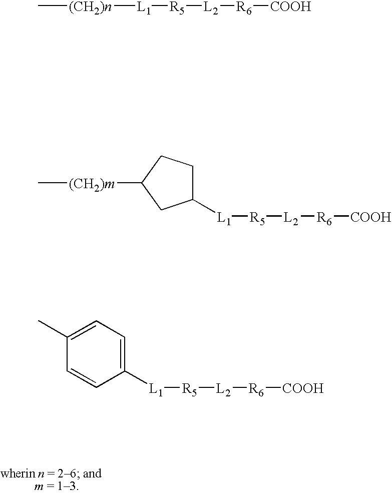
wherein
n=2-6; and
m=1-3.

6. A compound as claimed in claim 1, wherein L₁-R₅-L₂ is CO—NHR₅CO—NH and wherein NHR₅CO comprises a simple amino acid residue that contains a side-chain protonatable amine functionality.

7. A compound as claimed in claim 6 wherein NHR₅CO is represented by the formula:
—NH—CH[(CH₂)ₚN⁺R₈R₉R₁₀]CO—
wherein p is 1 to 5 and R₈, R₉ and R₁₀ are as defined above.

8. A compound as claimed in claim 7, wherein p is 1 to 4.

9. A compound as claimed in claim 1 wherein R₈, R₉ and R₁₀ are each independently C₁₋₄ alkyl.

10. A compound as claimed in claim 1 wherein L₂ is CONH; wherein R₆ is
—(CH₂)_{q}-Aₛ-(CH₂)ᵣ—;
where q and r are each 0 to 3, provided that both q and r are not both 0;
s is 0 or 1; and
A is a 5-10 membered stable monocyclic or bicyclic aromatic ring or a 3-6 membered carbocyclic or alicyclic ring.

11. A compound as claimed in claim 10 wherein r and s are 0 and q is 1 or 2.

12. A compound of general formula (Ia) as defined in claim 1.

13. A compound as claimed in claim 12,
wherein
X and Y are O,
R₁ is H; and
R₂ and R₃ are as defined in claim 1.

14. A compound as claimed in claim 13,
wherein:
R₃ is —(CH₂)ₒ—C(O)—NH—CH(—(CH₂)ₚ—N⁺(R₈)(R₉)(R₁₀))(—C(O)—R₆—COOH)
o is an integer from 2-6;
p is an integer from 1 to 5; and
R₆, R₈, R₉ and R₁₀ are as defined in claim 1.

15. A compound as claimed in claim 14, wherein p is an integer from 1 to 4.

16. A compound as claimed in claim 15,
wherein
R₃ is —(CH₂)ₒ—C(O)—NH—CH(—(CH₂)ₚ—N⁺(R₈)(R₉)(R₁₀))(—C(O)—R₆—COOH)
o is an integer from 3 to 6;
p is an integer from 2 to 4;
R₈ and R₉ are methyl; and
R₁₀ ═H or methyl.

17. A process for the preparation of a compound of general formula (I) in which L₁ and L₂ are CONH, the process comprising:
(i) reacting a compound of general formula V:
H₂N—R₆—COOH (V)
wherein R₆ is as defined for general formula (I) in claim 1; and
wherein the compound of general formula (V) is bound at its C-terminus to a solid support;
with a compound of general formula (VI):
W—NH—R₅—COOH (VI)
wherein:
R₅ is as defined in claim 1; and
W is a protecting group;
(ii) removal of the protecting group W and reaction with a compound of general formula (VII): 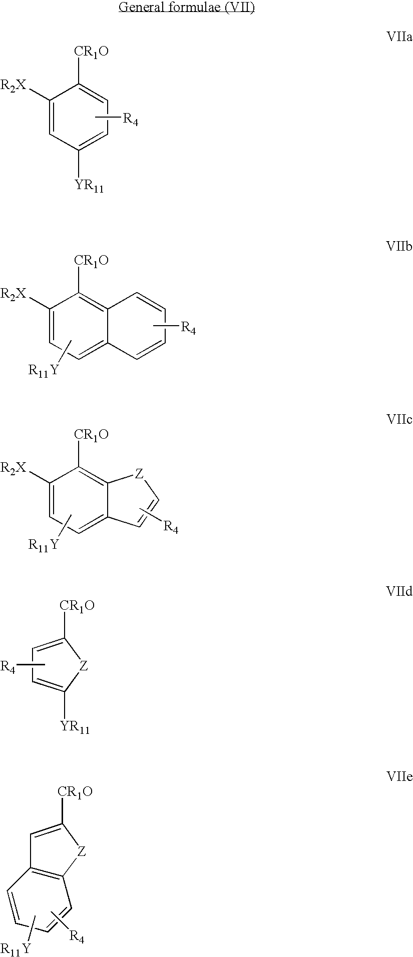
wherein
X, Y, Z, R₁, R₂, and R₄ are as defined in claim 1; R₁₁ is C₁₋₇ alkyl-COOH, C₃₋₁₀ cycloalkyl-COOH or Ar—C₀₋₇ alkyl-COOH; and
(iii) removal of the product from the solid support.

18. A process as claimed in claim 17 wherein, in the compound of general formula (VI), W is a urethane protecting group.

19. A compound of general formula (XIV): 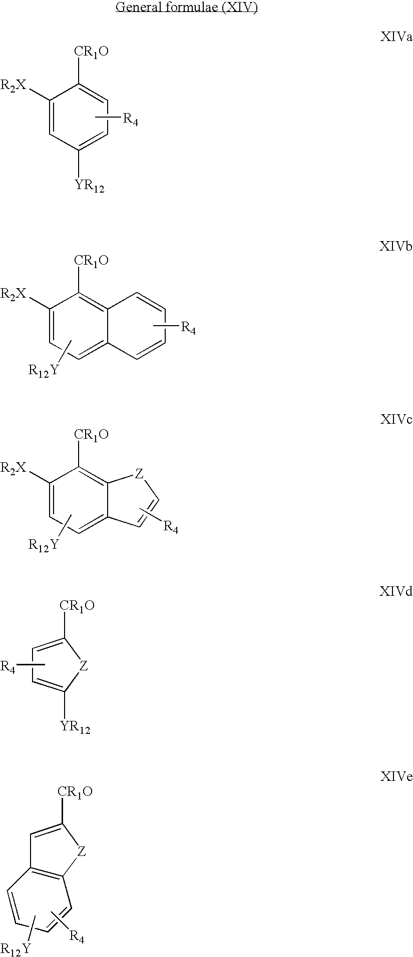
wherein
X, Y, Z, R₁, R₂, and R₄ are as defined in claim 1; and
R₁₂ is C₁₋₇ alkyl-L₁-R₅-L₂-R₆CONHQ, C₃₋₁₀ cycloalkyl-L₁-R₅-L₂-R₆CONHQ or Ar—C₀₋₇ alkyl-L₁-R₅-L₂-R₆CONH-Q;
wherein L₁, L₂, R₅, and R₆ are as defined in claim 1;
Q is a part of a carrier and Q contains at least one amino group; and
wherein the carrier may contain more than one Q.

20. A compound as claimed in claim 19 wherein Q is part of a proteinaceous molecule, a polysaccharide, cellulose beads, a polymeric amino acid, a polymer, which may be a copolymer, an inactive virus particle or attenuated bacteria.

21. A process for the preparation of a compound as claimed in claim 19, the process comprising reacting a compound of general formula (I) as defined above with a carrier.

22. A compound of general formula (XV): 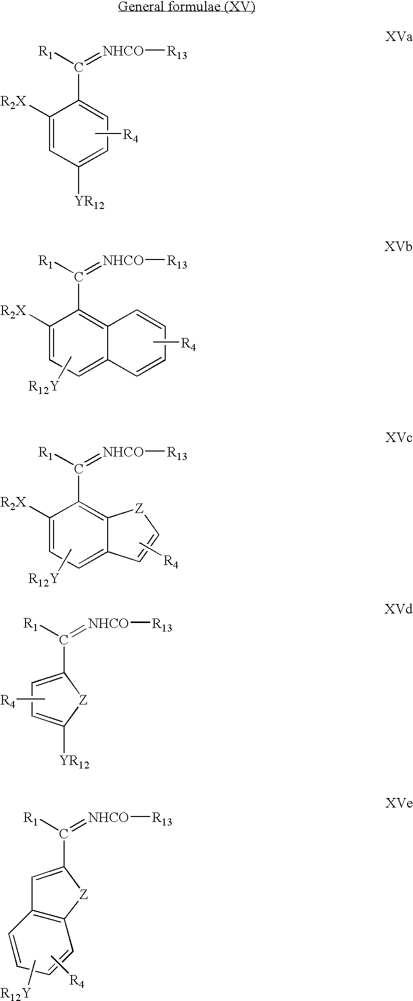
wherein X, Y, Z, R₁, R₂, and R₄ are as defined in claim 1;
R₁₂ is as defined in claim 19;
R₁₃ is (CH₂)ₜCONH-E, CONH-E, or G;
t is an integer from 1 to 5;
E is derived from an active moiety which either contains an amino group or has been derivatized to do so; and NHE is derived from the amino group of the active moiety;
G is an active moiety bound to the carbonylhydrazide through a carbon atom.

23. A compound as claimed in claim 22 which comprises E or G groups derived from two or more active moieties.

24. A process for the preparation of a compound of general formula (XV) as defined in claim 22, the process comprising reacting a compound of general formula (XIV) as defined above with a compound of general formula (XVIa), (XVIb) or (XVIc):
E-NH—CO—(CH₂)ₜCONHNH₂ (XVIa)
E-NH—CO—NHNH₂ (XVIb)
G-CO—NHNH₂ (XVIc)
where E, G and t are as defined in claim 22.

25. A compound as claimed in claim 22 which is soluble in aqueous solution.

26. A compound as claimed in claim 25 wherein E or G is derived from an epitope or mimotope.

27. A compound as claimed in claim 26 wherein the epitope is a fragment derived from a protein or peptide molecule or a variant thereof.

28. A compound as claimed in claim 26 wherein the epitope is a B cell or T cell epitope.

29. A compound as claimed in claim 25 which includes another active moiety comprising an immunomodulating compound attached to the carrier protein.

30. A method for raising specific antibodies against an epitope or mimotope, the method comprising immunizing a subject with a compound as claimed in claim 26 comprising E or G derived from said epitope or mimotope.

31. (canceled)

32. (canceled)

33. (canceled)

34. A pharmaceutical composition comprising a compound as claimed in claim 26 together with a pharmaceutically acceptable excipient.

35. A vaccine composition comprising a compound as claimed in claim 26 together with a pharmaceutically acceptable excipient and a pharmaceutically acceptable adjuvant.

36. A non-destructive method of quantifying the extent and/or rate of reaction of a protein with a linker of general formula (I) as defined in claim 1 in which R₂ is H and X is O, the method comprising either:
a) measuring the intensity of the absorbance spectrum at a wavelength above 300 nm and at a pH greater than 7 in order to detect the formation of a compound of general formula (XIV) in which R₂ is H and X is O; or
b) measuring the fluorescence emission upon excitation at a selected wavelength in order to detect the formation of a compound of general formula (XIV) as defined in claim 19 in which R₂ is Hand X is O.

37. A non-destructive method for quantifying the extent and/or rate of reaction of a linker-protein of general formula (XIV) as defined in claim 19 wherein R₂ is H and X is O, with an active moiety hydrazide, the process comprising measuring the intensity of the absorbance spectrum at a wavelength above 300 nm and a pH less than 7.

38. A process for the preparation of a compound of general formula (XV) as defined in claim 22 in which:
R₂ is Hand X is O;
the carrier has more than one Q;
a first selected percentage of the Q groups is derivatized with a first active moiety; and, optionally
further selected percentages of the Q groups are derivatized with further active moieties;
the process comprising:
a) reacting a compound of general formula (XIV) as defined in claim 19 in which R₂ is H and X is O with a first compound of general formula (XVI) as defined in claim 24 at a pH less than 7;
b) monitoring the progress of the reaction by measuring the intensity of the absorbance spectrum at a wavelength of above 300 nm and stopping the reaction when the intensity of the absorbance spectrum reaches the first selected percentage of the known maximum intensity; and optionally
c) reacting the product of steps (a) and (b) with one or more further compounds of general formula (XVI), monitoring the progess of the reaction by measuring the intensity of the absorbance spectrum at a wavelength of above 300 nm and stopping the reaction when the intensity of the absorbance spectrum reaches further selected percentages of the known maximum intensity.

39. A method for quantifying the extent and/or rate of release of an active moiety hydrazide from a compound of general formula (XV) as defined in claim 22 in which R₂ is H and X is O, the method comprising the measurement of the absorbance spectrum maximum at a wavelength above 300 nm and at pH less than 7.

40. A compound as claimed in claim 22 wherein E or G is a labelling moiety.

41. A compound as claimed in claim 22 which is insoluble in aqueous solution.

42. A compound as claimed in claim 41 wherein E or G is a ligand which is specific for a compound to be separated.

43. A compound as claimed in claim 42 which contains additional E or G groups derived from labelling molecules.

44. A method of separating a compound from a mixture, the method comprising contacting the mixture with a compound of claim 42 wherein E or G is a ligand which is specific for said compound and the carrier is a solid support.

45. An assay method comprising contacting a mixture suspected of containing an analyte with a compound of claim 55 in which E or G is a ligand for said analyte and the carrier is a solid support.

46. A wound dressing comprising a compound as claimed in claim 41.

47. (canceled)

48. (canceled)

49. (canceled)

50. A compound as claimed in claim 41 wherein the carrier is a polymer suitable for use in dialysis tubing and E or G is heparin for use in the preparation of dialysis tubing.

51. Dialysis tubing comprising a compound as claimed in claim 50.

52. A compound as claimed in claim 26 wherein the epitope is an antigenic determinant derived from a protein or peptide molecule.

53. A compound as claimed in claim 29 wherein the immunomodulating compound is a lipid, an adjuvant, an immunostimulating DNA sequence or cytokine.

54. A compound as claimed in claim 25 wherein E or G is a labelling moiety.

55. A compound as claimed in claim 41 wherein E or G is a ligand which is specific for an analyte.

56. A compound as claimed in claim 41 wherein the carrier is a functionalized polymer of the type commonly used in wound dressings and E or G is a peptide growth factor, a chemo-attractant protein, a ligand or an analogue of one of these.
